(19) United States
(12) Patent Application Publication
(10) Pub. No.: US 2026/0252804 A1
(43) Pub. Date: Aug. 27, 2026

(54) SYSTEM

(71) Applicant: SoftBank Group Corp., Tokyo (JP)

(72) Inventor: Yoko HANYU, Tokyo (JP)

(73) Assignee: SoftBank Group Corp., Tokyo (JP)

(21) Appl. No.: 19/536,257

(22) Filed: Feb. 11, 2026

(30) Foreign Application Priority Data
Feb. 21, 2025 (JP) 2025-026994

(51) Int. Cl. G06F 40/30

(52) U.S. Cl. CPC G06F 40/30

(57) ABSTRACT

The system according to the embodiment comprises a speaking unit, a conversation receiving unit, a summarizing unit, a storage unit, and a confirmation unit. The speaking unit is configured to speak to a subject. The conversation receiving unit is configured to receive a conversation from the subject spoken to by the speaking unit. The summarizing unit is configured to summarize the conversation received by the conversation receiving unit. The storage unit is configured to store the content summarized by the summarizing unit. The confirmation unit is configured to confirm the content stored by the storage unit.

### CROSS-REFERENCE TO RELATED APPLICATION

The present application claims priority to and incorporates by reference the entire contents of Japanese Patent Application No. 2025-026994 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, monitoring of elderly people living apart has not been sufficiently performed, and there is room for improvement.

### SUMMARY OF THE INVENTION

The system according to the embodiment comprises a speaking unit, a conversation receiving unit, a summarizing unit, a storage unit, and a confirmation unit. The speaking unit is configured to speak to a subject. The conversation receiving unit is configured to receive a conversation from the subject spoken to by the speaking unit. The summarizing unit is configured to summarize the conversation received by the conversation receiving unit. The storage unit is configured to store the content summarized by the summarizing unit. The confirmation unit is configured to confirm the content stored by the storage unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

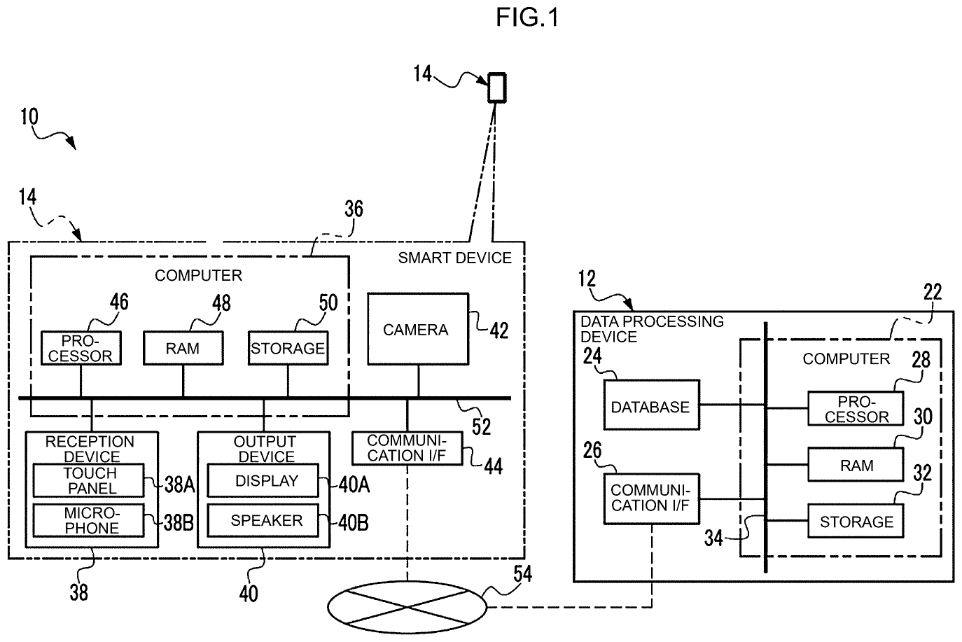

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

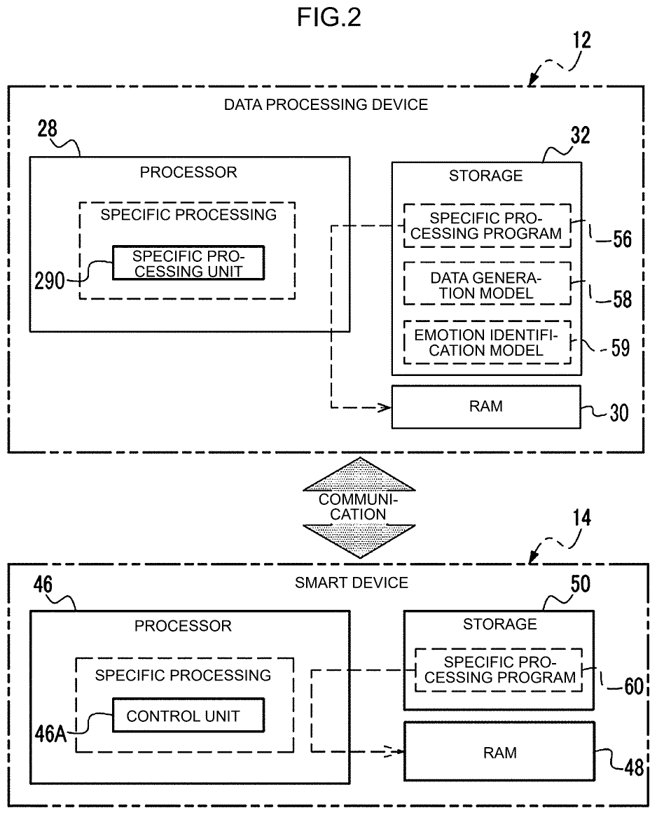

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The monitoring system according to the embodiment of the present invention is a system that utilizes generative AI to monitor elderly parents living apart. This monitoring system has generative AI speak to the subject (elderly parent) by voice at a fixed time every day, and the subject begins a conversation with the generative AI. The generative AI summarizes the content of the conversation and saves it on the server as a conversation memo for the day. The service user (for example, a child living apart) can check the conversation at any desired timing and monitor for any abnormalities. For example, the generative AI speaks to the subject by voice at a fixed time every day, such as, “Good morning. What plans do you have today?” This allows the subject to communicate with the generative AI on a daily basis. Next, the subject starts a conversation with the generative AI, such as, “I plan to have lunch with a friend today.” The generative AI analyzes this conversation in real time and extracts important information. The generative AI summarizes the content of the conversation and saves it on the server as a conversation memo for the day. For example, a summary such as “The subject plans to have lunch with a friend” is generated. This summary is saved on the server so that the service user can check it later. The service user can check the conversation at any desired timing and monitor for any abnormalities. For example, if the subject behaves differently than usual or seems to be in poor health, abnormalities can be detected early. This system enables monitoring of elderly parents living apart and provides peace of mind. Furthermore, by utilizing generative AI, communication with the subject is smooth, and daily monitoring becomes easier. As a result, the monitoring system enables monitoring of elderly parents living apart and provides peace of mind. Specifically, this monitoring system is composed of multiple computer modules, including a natural language processing engine centered on a large language model, a speech recognition module, a speech synthesis module, a conversation summarization module, an anomaly detection module, a database management module, and a user interface module. The system first has the speech recognition module acquire the subject's spoken voice in 16 kHz/16 bit PCM format, perform preprocessing such as spectrogram conversion and noise removal, and input it to the speech recognition engine (e.g., Transformer-based ASR model). The speech recognition engine takes the speech waveform tensor (e.g., shape=[T, 80] mel spectrogram) as input and generates a sequence of spoken text (e.g., “I plan to have lunch with a friend today”) as output. Next, the natural language processing engine tokenizes the spoken text sequence and converts it into context vectors (e.g., shape=[N, 768] embedding vectors). The large language model takes these context vectors as input and extracts a summary of the conversation (e.g., “The subject plans to have lunch with a friend”) and important keywords (e.g., “lunch,” “friend”). Furthermore, the anomaly detection module compares the conversation summary data for the past 30 days (e.g., a list of daily summary texts) with the summary text for the current day and calculates an anomaly score (e.g., 0.85) using cosine similarity in vector space and clustering methods (e.g., K-means). If the anomaly score exceeds a predetermined threshold (e.g., 0.7), an anomaly flag is set and the service user is notified. The results of conversation summarization and anomaly detection are saved on the server as structured data, including date, time, summary text, anomaly score, etc., by the database management module. The service user can view the saved conversation summaries and anomaly detection results in chronological order via the user interface module. As a technical effect, this system eliminates the need for manual sequential conversation recording and anomaly monitoring, and enables efficient, accurate, and early anomaly detection in monitoring operations by realizing high-precision natural language understanding, summarization, and anomaly detection by AI in real time and automatically. In addition, by utilizing parallel computing clusters with GPUs and distributed databases, large volumes of conversation data can be processed and stored quickly and stably. Specific application fields include elderly monitoring services, remote caregiving support, home medical monitoring, support for people with disabilities, and child safety monitoring.

The monitoring system according to the embodiment comprises a speaking unit, a conversation receiving unit, a summarizing unit, a storage unit, and a confirmation unit. The speaking unit speaks to the subject. For example, the speaking unit speaks to the subject by voice at a fixed time every day, such as, “Good morning. What plans do you have today?” This allows the subject to communicate with the generative AI on a daily basis. The conversation receiving unit receives a conversation from the subject spoken to by the speaking unit. For example, the conversation receiving unit receives the content of the subject's reply to the speaking unit, such as, “I plan to have lunch with a friend today.” The summarizing unit summarizes the conversation received by the conversation receiving unit. For example, the summarizing unit uses generative AI to analyze the content of the subject's conversation in real time and extract important information. The generative AI generates a summary such as, “The subject plans to have lunch with a friend.” The storage unit stores the content summarized by the summarizing unit. For example, the storage unit saves the summary generated by the generative AI on the server. The confirmation unit confirms the content stored by the storage unit. For example, the confirmation unit allows the service user to check the summary saved on the server at any desired timing. As a result, the monitoring system according to the embodiment can efficiently manage, store, and confirm conversations with the subject. Specifically, this monitoring system is composed of multiple computer modules, including a speech recognition module, a natural language processing engine, a conversation summarization module centered on a large language model, an anomaly detection module, a database management module, and a user interface module. The system first has the speaking unit use a speech synthesis module to generate spoken voice such as “Good morning. What plans do you have today?” as 16 kHz/16 bit PCM audio data, and outputs it to the subject via a speaker device. The conversation receiving unit receives the subject's spoken voice acquired from a microphone in real time, performs preprocessing such as noise removal and spectrogram conversion, and inputs it to the speech recognition engine (e.g., Transformer-based ASR model). The speech recognition engine takes the speech waveform tensor (e.g., shape=[T, 80] mel spectrogram) as input and generates a sequence of spoken text (e.g., “I plan to have lunch with a friend today”) as output. The summarizing unit uses the natural language processing engine to tokenize the spoken text sequence, convert it into context vectors (e.g., shape=[N, 768]), and then inputs it to the large language model (e.g., Transformer architecture). The large language model extracts a summary of the conversation (e.g., “The subject plans to have lunch with a friend”) and important keywords (e.g., “lunch,” “friend”), and the summarizing unit outputs these as structured data. The storage unit saves the summary text and keywords received from the summarizing unit on the server via the database management module, together with metadata such as date, time, and conversation ID. The confirmation unit allows the service user to view the saved conversation summaries and keywords in chronological order via the user interface module. As a technical effect, this system eliminates the need for manual sequential conversation recording and summarization, and enables efficient, accurate, and early anomaly detection in monitoring operations by realizing high-precision speech recognition, natural language understanding, and summary generation by AI in real time and automatically. In addition, by utilizing parallel computing clusters with GPUs and distributed databases, large volumes of conversation data can be processed and stored quickly and stably. Specific application fields include elderly monitoring services, remote caregiving support, home medical monitoring, support for people with disabilities, and child safety monitoring.

An emotion analysis unit configured to perform emotion analysis is provided. The emotion analysis unit analyzes the emotion of the subject. For example, the emotion analysis unit analyzes the tone of the subject's voice and facial expressions to estimate emotion. The emotion analysis unit detects, for example, a lower tone of voice when the subject is sad. The emotion analysis unit also detects a higher tone of voice when the subject is happy. Thus, by analyzing the subject's emotion, the emotion analysis unit enables more appropriate responses. Specifically, the emotion analysis unit is composed of multiple computer modules, including a speech recognition module, an image analysis module, a natural language processing engine, and a multimodal large language model. The emotion analysis unit first has the speech recognition module acquire the subject's spoken voice data (e.g., 16 kHz/16 bit PCM format, shape=[T] waveform array), perform preprocessing such as spectrogram conversion and noise removal, and extract acoustic features (e.g., F0, MFCC, energy, spectral envelope, etc.). The image analysis module takes facial image frames acquired from a camera (e.g., shape=[H, W, 3] RGB image) as input and performs face detection, landmark extraction, and facial expression classification (e.g., smile, sadness, surprise, etc.) using neural networks such as CNNs or Vision Transformers. The natural language processing engine tokenizes the spoken text sequence (e.g., “I'm very happy today”) and converts it into context vectors (e.g., shape=[N, 768]). The multimodal large language model integrates acoustic feature vectors, facial expression feature vectors, and spoken text embedding vectors, and outputs emotion labels (e.g., joy, sadness, anger, surprise, etc.), emotion scores (e.g., joy 0.85, sadness 0.05, etc.), and time-series emotion transition data (e.g., emotion score array for the past 10 minutes). Input examples include: (1) a bright voice saying “I plan to have lunch with a friend today” plus a smiling face image; (2) a depressed voice saying “I've been a bit tired lately” plus a neutral face image; (3) a flat voice saying “Nothing in particular has changed” plus a normal face image. Output examples include: (1) emotion label “joy,” score 0.92; (2) emotion label “fatigue,” score 0.78; (3) emotion label “neutral,” score 0.65. Based on these output results, the emotion analysis unit notifies subsequent modules such as the speaking unit or relaxation suggestion unit of the emotional state, and automatically adjusts response content, timing, and intervention methods. As a technical effect, the emotion analysis unit realizes integrated analysis of multidimensional features that is difficult with human subjective judgment or simple rule-based processing, and performs high-precision, real-time estimation of complex emotions from speech, facial expressions, and text, thereby greatly improving the response accuracy, flexibility, and user satisfaction of the entire monitoring system. In addition, by utilizing parallel computing with GPUs and distributed inference infrastructure, large volumes of multimodal data can be processed quickly. Specific application fields include elderly monitoring services, remote caregiving support, home medical monitoring, support for people with disabilities, child safety monitoring, mental health care, and customer support.

An anomaly detection unit configured to detect anomalies by comparing with past conversation history is provided. The anomaly detection unit detects anomalies by comparing with past conversation history. For example, the anomaly detection unit saves the subject's past conversation history in a database and compares it with the current conversation content. The anomaly detection unit can detect abnormalities early, for example, when the subject behaves differently than usual or seems to be in poor health. Thus, by detecting anomalies through comparison with past conversation history, the anomaly detection unit enables early detection of abnormalities. Specifically, the anomaly detection unit is composed of multiple computer modules, including a database management module, a natural language processing engine, a large language model, an anomaly score calculation module, and a notification module. The anomaly detection unit first has the database management module acquire conversation summary data for the past 30 days (e.g., a list of daily summary texts, shape=[30, L] text array) and the current conversation content (e.g., latest spoken text sequence, shape=[M]), and inputs them to the natural language processing engine. The natural language processing engine tokenizes each summary text and converts it into context vectors (e.g., shape=[N, 768]). The large language model (e.g., Transformer architecture) takes these vectors as input and performs feature extraction and anomaly score estimation for the conversation content. The anomaly score calculation module calculates an anomaly score (e.g., 0.85) using cosine similarity and clustering methods (e.g., K-means, DBSCAN, etc.) between the past conversation summary vectors and the current summary vector. Input examples include: (1) a normal utterance such as “I plan to have lunch with a friend today”; (2) an abnormal utterance such as “I felt sick and stayed in bed all day today”; (3) an isolated utterance such as “I haven't talked to anyone recently.” Output examples include: (1) anomaly score 0.15 (normal); (2) anomaly score 0.82 (abnormal); (3) anomaly score 0.91 (strong abnormality). If the anomaly score exceeds a predetermined threshold (e.g., 0.7), the notification module sets an anomaly flag and automatically notifies the service user. Subsequent processing includes not only displaying the anomaly detection result on the dashboard, but also, as needed, automatic contact with care staff or family, and proposals for additional health checks. As a technical effect, the anomaly detection unit realizes integrated analysis of high-dimensional features that is difficult with human subjective judgment or simple rule-based processing, and detects subtle changes and abnormal tendencies in conversation content with high precision and in real time, thereby greatly improving the anomaly detection accuracy, response speed, and user peace of mind of the entire monitoring system. In addition, by utilizing parallel computing with GPUs and distributed inference infrastructure, large volumes of conversation data can be processed quickly. Specific application fields include elderly monitoring services, remote caregiving support, home medical monitoring, support for people with disabilities, child safety monitoring, and mental health care.

A behavior monitoring unit configured to monitor the behavior of the subject is provided. The behavior monitoring unit monitors the behavior of the subject. For example, the behavior monitoring unit monitors the subject's behavior using cameras or sensors. The behavior monitoring unit issues an alert, for example, when the subject does not move for a certain period of time. Thus, by monitoring the subject's behavior, the behavior monitoring unit enables early detection of abnormal behavior. Specifically, the behavior monitoring unit is composed of multiple computer modules, including an image analysis module, a motion sensor data acquisition module, a behavior feature extraction module, an abnormal behavior determination module, and a notification module. The behavior monitoring unit first takes as input continuous image frames acquired from a camera (e.g., shape=[T, H, W, 3] RGB video data) and time-series sensor data acquired from accelerometers, gyroscopes, etc. (e.g., shape=[T, 3] acceleration vector). The image analysis module uses neural networks such as CNNs or Vision Transformers to perform posture estimation, motion classification, and region detection of the subject. The behavior feature extraction module integrates image features and sensor features to calculate behavior labels (e.g., walking, sitting, falling, stationary, etc.) and behavior scores (e.g., stationary score 0.95). The abnormal behavior determination module sets an abnormal flag when a stationary state continues for a certain period (e.g., 30 minutes) or when a falling motion is detected. Input examples include: (1) images of sitting without movement for 30 minutes plus stationary acceleration data; (2) images including a falling motion plus sudden acceleration change; (3) images of normal walking motion plus stable acceleration data. Output examples include: (1) abnormal label “long-term stationary”; (2) abnormal label “fall”; (3) normal label “walking.” When an abnormality is detected, the notification module automatically notifies the service user or care staff, and subsequent processing such as on-site confirmation or emergency response instructions is executed. As a technical effect, the behavior monitoring unit realizes high-precision, real-time detection of complex behavior patterns and abnormal signs that are difficult with human visual monitoring or simple motion detection, by AI-based multidimensional feature analysis, thereby greatly improving the safety, response speed, and operational efficiency of the entire monitoring system. In addition, by utilizing parallel computing with GPUs and distributed inference infrastructure, large volumes of video and sensor data can be processed quickly. Specific application fields include elderly monitoring services, remote caregiving support, home medical monitoring, support for people with disabilities, child safety monitoring, and safety monitoring of factory workers.

A keyword extraction unit configured to extract important keywords is provided. The keyword extraction unit extracts important keywords from the content of the conversation. For example, the keyword extraction unit uses generative AI to extract frequently occurring words and contextually important words from the conversation. The keyword extraction unit extracts keywords, for example, when the subject says a keyword such as “I am not feeling well.” Thus, by extracting important keywords, the keyword extraction unit makes it easier to grasp the main points of the conversation. Specifically, the keyword extraction unit is composed of multiple computer modules, including a natural language processing engine, a large language model, an importance score calculation module, a keyword ranking module, and a database management module. The keyword extraction unit first inputs the conversation text sequence (e.g., shape=[L] token sequence) to the natural language processing engine and performs morphological analysis and tokenization. The large language model (e.g., Transformer architecture) generates context vectors (e.g., shape=[N, 768]) and extracts candidate important keywords using attention weights, TF-IDF scores, and contextual features. The importance score calculation module calculates an importance score (e.g., 0.92) for each keyword based on frequency, contextual relevance, comparison with past conversation history, etc., and the keyword ranking module selects the top keywords. Input examples include: (1) utterances such as “I am not feeling well today” and “I forgot to take my medicine”; (2) utterances such as “lunch with a friend” and “going to the park”; (3) utterances such as “Nothing in particular has changed.” Output examples include: (1) keywords “condition,” “medicine,” “bad”; (2) keywords “friend,” “lunch,” “park”; (3) keywords “change,” “none.” The extracted keywords are used in subsequent processing such as summarization, anomaly detection, and dashboard display. As a technical effect, the keyword extraction unit realizes high-precision, real-time extraction of context-dependent important words that is difficult with human subjective judgment or simple rule-based processing, by AI-based high-dimensional feature analysis, enabling automatic grasping of conversation main points, early detection of abnormal signs, and improved information searchability. In addition, by utilizing parallel computing with GPUs and distributed inference infrastructure, large volumes of conversation data can be processed quickly. Specific application fields include elderly monitoring services, remote caregiving support, home medical monitoring, support for people with disabilities, customer support, and automatic FAQ generation.

The speaking unit is capable of setting a speaking time in accordance with the subject's daily rhythm. The speaking unit sets the speaking time in accordance with the subject's daily rhythm. For example, the speaking unit flexibly adjusts the speaking time by considering the subject's daily activity patterns and sleep times. Thus, by setting the speaking time in accordance with the subject's daily rhythm, the speaking unit enables more natural communication. Specifically, the speaking unit is composed of multiple computer modules, including a behavior history analysis module, a sleep detection module, a scheduling engine, a speech synthesis module, and a user interface module. The speaking unit first has the behavior history analysis module acquire activity logs for the past week (e.g., timestamp data for wake-up time, bedtime, meal time, outing time, etc., shape=[7, N]), and the sleep detection module acquires sleep time and sleep quality data (e.g., shape=[7, 2]) from wearable sensors or bed sensors. The scheduling engine estimates the subject's daily rhythm by time-series analysis (e.g., autoregressive model, LSTM, etc.) based on these data and automatically determines the optimal speaking timing (e.g., 30 minutes after waking up, before lunch, before bedtime, etc.). The speech synthesis module generates spoken voice such as “Good morning. What plans do you have today?” at the determined timing and outputs it via a speaker device. Input examples include: (1) a regular daily pattern of waking up at 7:00 and going to bed at 22:00; (2) an irregular pattern with late wake-up and naps only on weekends; (3) irregular sleep and activity patterns. Output examples include: (1) speaking at 7:30 every day; (2) speaking at 9:00 on weekends; (3) speaking immediately after activity is detected. Subsequent processing includes logging the speaking timing and automatic schedule adjustment based on user feedback. As a technical effect, the speaking unit realizes high-precision, automatic individual optimization and dynamic adjustment that is difficult with manual settings or simple time specification, by AI-based time-series analysis and pattern recognition, thereby greatly improving the naturalness of communication, user satisfaction, and response rate. In addition, by utilizing parallel computing with GPUs and distributed inference infrastructure, scheduling for many subjects can be processed quickly. Specific application fields include elderly monitoring services, remote caregiving support, home medical monitoring, support for people with disabilities, and personal assistants.

The behavior monitoring unit is capable of issuing an alert when the subject does not move for a certain period of time. The behavior monitoring unit issues an alert when the subject does not move for a certain period of time. For example, the behavior monitoring unit issues an alert when the subject does not move for more than 30 minutes. Thus, by issuing an alert when the subject does not move for a certain period of time, the behavior monitoring unit enables early detection of abnormalities. Specifically, the behavior monitoring unit is composed of multiple computer modules, including a motion sensor data acquisition module, a stationary state determination algorithm, an alert generation module, a database management module, and a notification module. The behavior monitoring unit first inputs time-series data (e.g., shape=[T, 3] acceleration vector) acquired from accelerometers or gyroscopes to the stationary state determination algorithm. The stationary state determination algorithm determines whether the acceleration change is below a threshold (e.g., less than 0.01 G) for a certain period (e.g., 30 minutes), and if the stationary state continues, the alert generation module sets an abnormal flag. Input examples include: (1) no acceleration change for 30 minutes; (2) walking starts after 10 minutes of stationary state; (3) intermittent small movements. Output examples include: (1) alert issued; (2) no alert; (3) no alert. When an alert is issued, the notification module automatically notifies the service user or care staff, and subsequent processing such as on-site confirmation or emergency response instructions is executed. As a technical effect, the behavior monitoring unit realizes high-precision, real-time automatic detection and immediate notification of long-term stationary states that is difficult with human visual monitoring or simple timer settings, by AI-based time-series data analysis, thereby greatly improving the safety, response speed, and operational efficiency of the entire monitoring system. In addition, by utilizing parallel computing with GPUs and distributed inference infrastructure, behavior monitoring for many subjects can be processed quickly. Specific application fields include elderly monitoring services, remote caregiving support, home medical monitoring, support for people with disabilities, and safety monitoring of factory workers.

The emotion analysis unit is capable of analyzing the subject's emotion from the content of the conversation. The emotion analysis unit analyzes the subject's emotion from the content of the conversation. For example, the emotion analysis unit uses generative AI to analyze keywords and context in the conversation and estimate the subject's emotion. The emotion analysis unit analyzes the emotion as “joy,” for example, when the subject makes a statement such as “I'm very happy today.” Thus, by analyzing the subject's emotion from the content of the conversation, the emotion analysis unit enables more appropriate responses. Emotion estimation is realized, for example, by using an emotion engine or generative AI with emotion estimation functions. Generative AI may be a text generative AI (e.g., LLM) or a multimodal generative AI, but is not limited to these examples. Specifically, the emotion analysis unit is composed of multiple computer modules, including a speech recognition module, a natural language processing engine, a multimodal large language model, an emotion classification algorithm, and a time-series emotion transition analysis module. The emotion analysis unit first has the speech recognition module acquire the subject's spoken voice data (e.g., 16 kHz/16 bit PCM format, shape=[T] waveform array), perform preprocessing such as spectrogram conversion and noise removal, and extract acoustic features (e.g., fundamental frequency F0, mel-frequency cepstral coefficients MFCC, energy, spectral envelope, etc.). The natural language processing engine tokenizes the spoken text sequence (e.g., “I'm very happy today”) and converts it into context vectors (e.g., shape=[N, 768]). The multimodal large language model integrates acoustic feature vectors and text embedding vectors, and uses Transformer-based architecture and attention mechanisms to output emotion labels (e.g., joy, sadness, anger, surprise, etc.) and emotion scores (e.g., joy 0.85, sadness 0.05, etc.). Input examples include: (1) a bright voice saying “I plan to have lunch with a friend today”; (2) a depressed voice saying “I've been a bit tired lately”; (3) a flat voice saying “Nothing in particular has changed.” Output examples include: (1) emotion label “joy,” score 0.92; (2) emotion label “fatigue,” score 0.78; (3) emotion label “neutral,” score 0.65. Based on these output results, the emotion analysis unit notifies subsequent modules such as the speaking unit or relaxation suggestion unit of the emotional state, and automatically adjusts response content, timing, and intervention methods. Internally, the model optimizes emotion classification accuracy using loss functions such as cross-entropy loss and MSE loss, and performs pre-training on diverse emotion datasets using supervised learning and transfer learning. As a technical effect, the emotion analysis unit realizes integrated analysis of multidimensional features that is difficult with human subjective judgment or simple rule-based processing, and performs high-precision, real-time estimation of complex emotions from speech and text, thereby greatly improving the response accuracy, flexibility, and user satisfaction of the entire monitoring system. In addition, by utilizing parallel computing with GPUs and distributed inference infrastructure, large volumes of speech and text data can be processed quickly. Specific application fields include elderly monitoring services, remote caregiving support, home medical monitoring, support for people with disabilities, child safety monitoring, mental health care, and customer support.

The anomaly detection unit is capable of detecting anomalies by comparing with past conversation history. The anomaly detection unit detects anomalies by comparing with past conversation history. For example, the anomaly detection unit saves the subject's past conversation history in a database and compares it with the current conversation content. The anomaly detection unit can detect abnormalities early, for example, when the subject behaves differently than usual or seems to be in poor health. Thus, by detecting anomalies through comparison with past conversation history, the anomaly detection unit enables early detection of abnormalities. Specifically, the anomaly detection unit is composed of multiple computer modules, including a database management module, a natural language processing engine, a large language model, an anomaly score calculation module, and a notification module. The anomaly detection unit first has the database management module acquire conversation summary data for the past 30 days (e.g., a list of daily summary texts, shape=[30, L] text array) and the current conversation content (e.g., latest spoken text sequence, shape=[M]), and inputs them to the natural language processing engine. The natural language processing engine tokenizes each summary text and converts it into context vectors (e.g., shape=[N, 768]). The large language model (e.g., Transformer architecture) takes these vectors as input and performs feature extraction and anomaly score estimation for the conversation content. The anomaly score calculation module calculates an anomaly score (e.g., 0.85) using cosine similarity and clustering methods (e.g., K-means, DBSCAN, etc.) between the past conversation summary vectors and the current summary vector. Input examples include: (1) a normal utterance such as “I plan to have lunch with a friend today”; (2) an abnormal utterance such as “I felt sick and stayed in bed all day today”; (3) an isolated utterance such as “I haven't talked to anyone recently.” Output examples include: (1) anomaly score 0.15 (normal); (2) anomaly score 0.82 (abnormal); (3) anomaly score 0.91 (strong abnormality). If the anomaly score exceeds a predetermined threshold (e.g., 0.7), the notification module sets an anomaly flag and automatically notifies the service user. Subsequent processing includes not only displaying the anomaly detection result on the dashboard, but also, as needed, automatic contact with care staff or family, and proposals for additional health checks. Internally, the model optimizes anomaly detection accuracy using supervised learning and self-supervised learning for anomaly detection, and loss functions such as binary cross-entropy and triplet loss for anomaly detection. As a technical effect, the anomaly detection unit realizes integrated analysis of high-dimensional features that is difficult with human subjective judgment or simple rule-based processing, and detects subtle changes and abnormal tendencies in conversation content with high precision and in real time, thereby greatly improving the anomaly detection accuracy, response speed, and user peace of mind of the entire monitoring system. In addition, by utilizing parallel computing with GPUs and distributed inference infrastructure, large volumes of conversation data can be processed quickly. Specific application fields include elderly monitoring services, remote caregiving support, home medical monitoring, support for people with disabilities, child safety monitoring, and mental health care.

The speaking unit is capable of estimating the subject's emotion and adjusting the content of the speech based on the estimated emotion. The speaking unit estimates the subject's emotion and adjusts the content of the speech based on the estimated emotion. For example, the speaking unit uses generative AI to estimate the subject's emotion. The speaking unit, for example, offers words of encouragement when the subject is sad, such as, “Are you okay? Is there anything I can help you with?” The speaking unit also offers words of empathy when the subject is happy, such as, “That's wonderful!” Thus, by adjusting the content of the speech based on the subject's emotion, the speaking unit enables more appropriate communication. Emotion estimation is realized, for example, by using an emotion engine or generative AI with emotion estimation functions. Generative AI may be a text generative AI (e.g., LLM) or a multimodal generative AI, but is not limited to these examples. Specifically, the speaking unit receives emotion estimation results (e.g., emotion label “sadness,” score 0.82, etc.) from the emotion analysis unit as input. The emotion analysis unit integrates spoken voice data acquired by the speech recognition module (16 kHz/16 bit PCM format, shape=[T] waveform array), facial image frames acquired by the image analysis module (shape=[H, W, 3] RGB image), and spoken text sequence generated by the natural language processing engine (e.g., “I'm feeling a bit lonely today”), and inputs them to a multimodal large language model (e.g., Transformer-based). The model integrates acoustic features (F0, MFCC, etc.), facial expression features (face landmarks, expression classification labels), and text embedding vectors (shape=[N, 768]), and outputs emotion labels (e.g., sadness, joy, anger, etc.) and emotion scores (e.g., sadness 0.82, joy 0.05, etc.). Input examples include: (1) a sad voice saying “I'm lonely today” plus a sad facial expression image; (2) a bright voice saying “I'm very happy” plus a smiling face image. Output examples include: (1) emotion label “sadness,” score 0.82; (2) emotion label “joy,” score 0.91. The speaking unit uses these emotion estimation results as prompts for the response generation module (large language model) to generate response sentences using response generation algorithms (e.g., conditional text generation, emotion-controlled response generation). For example, for a sadness label, the response sentence “Are you okay? Is there anything I can help you with?” is generated; for a joy label, “That's wonderful!” is generated, and the speech synthesis module outputs the response as 16 kHz/16 bit PCM audio data. Subsequent processing includes recording the generated response content in the conversation history database and using it for future conversation content and emotion transition analysis. As a technical effect, the speaking unit realizes integrated analysis of multidimensional features (speech, facial expression, text) and automatic optimization of response content according to emotion, which is difficult with human subjective judgment or simple rule-based processing, thereby greatly improving the naturalness of communication, user satisfaction, and psychological care effect. In addition, by utilizing parallel computing with GPUs and distributed inference infrastructure, real-time emotion-adaptive response generation for many subjects can be processed quickly. Specific application fields include elderly monitoring services, remote caregiving support, home medical monitoring, support for people with disabilities, mental health care, and customer support.

The speaking unit is capable of analyzing the subject's past conversation history and selecting the content of the speech. The speaking unit analyzes the subject's past conversation history and selects the optimal content of the speech. For example, the speaking unit provides topics related to hobbies that the subject has talked about in the past, such as, “How is your hobby going recently?” The speaking unit also provides topics related to health that the subject has talked about in the past, such as, “How is your health condition recently?” Furthermore, the speaking unit provides topics related to family that the subject has talked about in the past, such as, “How is your family doing?” Thus, by analyzing the subject's past conversation history, the speaking unit can select more appropriate content of the speech. Specifically, the speaking unit is composed of multiple computer modules, including a conversation history database reference module, a natural language processing engine, a conversation history feature extraction module, a topic selection algorithm, a response generation module, and a speech synthesis module. The speaking unit first has the conversation history database reference module acquire conversation history data for the past 30 days (e.g., structured data including date, time, spoken text, summary, emotion label, etc., shape=[30, L]). The natural language processing engine tokenizes each spoken text and converts it into context vectors (e.g., shape=[N, 768]). The conversation history feature extraction module performs topic clustering (e.g., K-means, LDA, etc.) and TF-IDF scoring on these vectors to extract frequently occurring topic categories (e.g., hobbies, health, family, etc.) and important keywords (e.g., “lunch,” “blood pressure,” “grandchild,” etc.). The topic selection algorithm comprehensively evaluates the frequency of extracted topic categories and keywords, recent conversation trends, and emotion transitions to automatically select the most relevant and interesting topic for the next speech. For example, if there have been many utterances about “gardening” or “walking” in the past week, a topic such as “How is your gardening going recently?” is generated. The response generation module inputs the selected topic and summary of past history as prompts to a large language model (e.g., Transformer architecture) and generates natural dialogue sentences (e.g., “How is your health condition recently?”). The speech synthesis module outputs the generated text as 16 kHz/16 bit PCM audio data. Input examples include: (1) conversation history data for the past 30 days (e.g., “I played with my grandchild last week,” “My blood pressure has been high recently,” etc.); (2) recent emotion transitions (e.g., frequent joy, frequent fatigue, etc.); (3) frequently occurring keyword lists (e.g., “walking,” “health,” “family,” etc.). Output examples include: (1) “How is your family doing recently?”; (2) “Is your health management going well?”; (3) “Have you started any new hobbies recently?” Subsequent processing includes recording the generated speech content in the conversation history database and using it for future topic selection and individual optimization. As a technical effect, the speaking unit realizes high-precision personalized topic recommendation based on past history, which is difficult with human subjective memory or simple random topic selection, by automating high-dimensional feature analysis, clustering, and natural language generation by AI, thereby greatly improving the affinity, continuity, and user satisfaction of communication. In addition, by utilizing parallel computing with GPUs and distributed inference infrastructure, analysis of conversation history and topic selection for many subjects can be processed quickly. Specific application fields include elderly monitoring services, remote caregiving support, home medical monitoring, support for people with disabilities, personal assistants, and customer support.

The speaking unit is capable of selecting a topic based on the subject's current activity status when speaking. The speaking unit selects a topic based on the subject's current activity status when speaking. For example, the speaking unit provides topics related to cooking when the subject is cooking, such as, “What are you making today?” The speaking unit provides topics related to walking when the subject is walking, such as, “Is your walk pleasant?” Furthermore, the speaking unit provides topics related to TV programs when the subject is watching TV, such as, “What program are you watching?” Thus, by selecting a topic based on the subject's current activity status when speaking, the speaking unit enables more appropriate communication. Specifically, the speaking unit is composed of multiple computer modules, including an activity status estimation module, a sensor data acquisition module, an image analysis module, an activity classification algorithm, a topic selection algorithm, a response generation module, and a speech synthesis module. The speaking unit first has the sensor data acquisition module acquire acceleration data (e.g., shape=[T, 3]), environmental sound data (e.g., shape=[T, 1]), camera images (e.g., shape=[H, W, 3]), etc., in real time from the subject's wearable devices or smart home sensors. The image analysis module uses neural networks such as CNNs or Vision Transformers to estimate activity labels such as “cooking,” “walking,” “watching TV,” etc., from images. The activity classification algorithm integrates sensor features (e.g., acceleration patterns, acoustic features, image features) to accurately determine the current activity category (e.g., cooking, walking, watching TV, resting, etc.). The topic selection algorithm selects highly relevant topic templates (e.g., “What are you making today?” “Is your walk pleasant?” etc.) according to the estimated activity category and inputs them as prompts to the response generation module (large language model). The response generation module generates natural dialogue sentences considering activity category, past history, emotion estimation results, etc., and the speech synthesis module outputs them as 16 kHz/16 bit PCM audio data. Input examples include: (1) walking pattern from acceleration data plus outdoor image→“walking” determination; (2) kitchen image plus cooking sound→“cooking” determination; (3) TV screen image plus stationary posture→“watching TV” determination. Output examples include: (1) “Where are you walking today?”; (2) “What kind of dish are you making?”; (3) “What program are you watching?” Subsequent processing includes recording activity estimation and topic selection results in the conversation history database and using them for future individual optimization and anomaly detection. As a technical effect, the speaking unit realizes real-time activity recognition and topic optimization that is difficult with human visual observation or simple schedule-based methods, by automating multidimensional sensor data analysis, image recognition, and natural language generation by AI, thereby greatly improving the immediacy, affinity, and user satisfaction of communication. In addition, by utilizing parallel computing with GPUs and distributed inference infrastructure, activity recognition and topic selection for many subjects can be processed quickly. Specific application fields include elderly monitoring services, remote caregiving support, home medical monitoring, support for people with disabilities, personal assistants, and smart homes.

The speaking unit is capable of estimating the subject's emotion and adjusting the timing of the speech based on the estimated emotion. The speaking unit estimates the subject's emotion and adjusts the timing of the speech based on the estimated emotion. For example, the speaking unit uses generative AI to estimate the subject's emotion. The speaking unit, for example, speaks during a relaxed time when the subject is relaxed, such as, “Let's talk during your relaxing time.” The speaking unit speaks during a time when the subject is not busy when the subject is busy, such as, “Let's talk avoiding your busy time.” Furthermore, the speaking unit speaks during a break when the subject is tired, such as, “Let's talk during your break.” Thus, by adjusting the timing of the speech based on the subject's emotion, the speaking unit enables more appropriate communication. Emotion estimation is realized, for example, by using an emotion engine or generative AI with emotion estimation functions. Generative AI may be a text generative AI (e.g., LLM) or a multimodal generative AI, but is not limited to these examples. Specifically, the speaking unit is composed of multiple computer modules, including an emotion analysis unit, an activity status estimation module, a scheduling engine, a response generation module, and a speech synthesis module. The speaking unit first has the emotion analysis unit use a speech recognition module, image analysis module, natural language processing engine, etc., to extract acoustic features, facial expression features, and text embedding vectors from the subject's spoken voice (e.g., 16 kHz/16 bit PCM waveform), facial image (e.g., shape=[H, W, 3]), and spoken text (e.g., “I've been a bit tired today”), and estimates emotion labels (e.g., relaxed, busy, fatigued, etc.) and emotion scores (e.g., relaxed 0.85, fatigue 0.78, etc.) using a multimodal large language model. The scheduling engine analyzes emotion estimation results and activity status (e.g., exercising, resting, working, etc.) in a time series and automatically determines the optimal speaking timing (e.g., during relaxation, during a break, after work, etc.). The response generation module generates dialogue sentences such as “Let's talk during your relaxing time,” “Let's talk avoiding your busy time,” etc., according to the determined timing, and the speech synthesis module outputs them as 16 kHz/16 bit PCM audio data. Input examples include: (1) emotion label “relaxed,” activity “resting”; (2) emotion label “busy,” activity “working”; (3) emotion label “fatigue,” activity “after exercise.” Output examples include: (1) “Let's talk during your relaxing time”; (2) “Let's talk avoiding your busy time”; (3) “Let's talk during your break.” Subsequent processing includes recording the speaking timing and emotion estimation results in the conversation history database and using them for future optimization and anomaly detection. Internally, the model performs supervised learning and transfer learning using cross-entropy loss and MSE loss to improve emotion classification accuracy. As a technical effect, the speaking unit realizes dynamic timing optimization according to emotion and activity status, which is difficult with human subjective judgment or simple time specification, by automating multidimensional feature analysis, time-series analysis, and natural language generation by AI, thereby greatly improving the naturalness of communication, user satisfaction, and response rate. In addition, by utilizing parallel computing with GPUs and distributed inference infrastructure, emotion-adaptive scheduling for many subjects can be processed quickly. Specific application fields include elderly monitoring services, remote caregiving support, home medical monitoring, support for people with disabilities, personal assistants, and mental health care.

The speaking unit is capable of selecting a highly relevant topic by considering the subject's geographic location when speaking. The speaking unit selects a highly relevant topic by considering the subject's geographic location when speaking. For example, the speaking unit provides topics related to parks when the subject is in a park, such as, “Is your walk in the park pleasant?” The speaking unit provides topics related to health when the subject is in a hospital, such as, “How was your medical examination at the hospital?” Furthermore, the speaking unit provides topics related to shopping when the subject is in a shopping mall, such as, “Is shopping fun?” Thus, by selecting a topic by considering the subject's geographic location when speaking, the speaking unit enables more appropriate communication. Specifically, the speaking unit is composed of multiple computer modules, including a location information acquisition module, a geographic context estimation module, a topic selection algorithm, a response generation module, and a speech synthesis module. The speaking unit first has the location information acquisition module acquire real-time latitude and longitude data (e.g., shape=[2]) from GPS sensors or smartphones. The geographic context estimation module matches the data with map databases and POI (Point of Interest) information to determine whether the current location corresponds to categories such as “park,” “hospital,” or “shopping mall.” The topic selection algorithm selects highly relevant topic templates (e.g., “Is your walk in the park pleasant?” “How was your medical examination at the hospital?” etc.) according to the estimated geographic category and inputs them as prompts to the response generation module (large language model). The response generation module generates natural dialogue sentences considering geographic context, past history, emotion estimation results, etc., and the speech synthesis module outputs them as 16 kHz/16 bit PCM audio data. Input examples include: (1) GPS data (latitude 35.6, longitude 139.7) plus POI “park”→“park” determination; (2) GPS data plus POI “hospital”→“hospital” determination; (3) GPS data plus POI “shopping mall”→“shopping mall” determination. Output examples include: (1) “Is your walk in the park pleasant?”; (2) “How was your medical examination at the hospital?”; (3) “Is shopping fun?” Subsequent processing includes recording geographic context and topic selection results in the conversation history database and using them for future individual optimization and anomaly detection. As a technical effect, the speaking unit realizes real-time location-linked topic optimization that is difficult with manual confirmation or simple time/history-based methods, by automating geographic context estimation and natural language generation by AI, thereby greatly improving the immediacy, affinity, and user satisfaction of communication. In addition, by utilizing parallel computing with GPUs and distributed inference infrastructure, location-linked topic selection for many subjects can be processed quickly. Specific application fields include elderly monitoring services, remote caregiving support, home medical monitoring, support for people with disabilities, personal assistants, and location-linked services.

The speaking unit is capable of analyzing the subject's social media activity when speaking and selecting a relevant topic. The speaking unit analyzes the subject's social media activity when speaking and selects a relevant topic. For example, the speaking unit provides topics related to photos shared by the subject on social media, such as, “That's a wonderful photo!” The speaking unit provides topics related to comments made by the subject on social media, such as, “Please tell me more about that comment.” Furthermore, the speaking unit provides topics related to accounts followed by the subject on social media, such as, “What do you think about that account?” Thus, by analyzing the subject's social media activity, the speaking unit can select more appropriate topics. Specifically, the speaking unit is composed of multiple computer modules, including a social media data acquisition module, a natural language processing engine, an image analysis module, a topic extraction algorithm, a response generation module, and a speech synthesis module. The speaking unit first has the social media data acquisition module acquire the latest post data (e.g., text posts, images, comments, list of followed accounts, etc.) via API or similar, with the subject's permission. The natural language processing engine tokenizes post text and comments and converts them into context vectors (e.g., shape=[N, 768]). The image analysis module analyzes post images (e.g., shape=[H, W, 3]) using CNNs or Vision Transformers to estimate image content (e.g., landscape, food, people, etc.) and emotion labels. The topic extraction algorithm integrates features of post text, images, comments, and followed accounts to extract the latest interests and topic categories (e.g., travel, hobbies, health, etc.). The response generation module inputs the extracted topics, image content, comment content, etc., as prompts to a large language model and generates natural dialogue sentences such as “That's a wonderful photo!” “Please tell me more about that comment,” “What do you think about that account?” The speech synthesis module outputs the generated text as 16 kHz/16 bit PCM audio data. Input examples include: (1) latest post image “cherry blossom photo” plus text “I went to the park”; (2) comment “I went to a new restaurant”; (3) followed account “culinary expert.” Output examples include: (1) “That's a wonderful photo!”; (2) “Please tell me more about that comment”; (3) “What do you think about that account?” Subsequent processing includes recording social media activity and topic selection results in the conversation history database and using them for future individual optimization and anomaly detection. As a technical effect, the speaking unit realizes real-time, multimodal social media-linked topic optimization that is difficult with manual confirmation or simple history-based methods, by automating natural language and image analysis, feature integration, and natural language generation by AI, thereby greatly improving the immediacy, affinity, and user satisfaction of communication. In addition, by utilizing parallel computing with GPUs and distributed inference infrastructure, social media-linked topic selection for many subjects can be processed quickly. Specific application fields include elderly monitoring services, remote caregiving support, home medical monitoring, support for people with disabilities, personal assistants, and customer support.

The conversation receiving unit is capable of estimating the subject's emotion and adjusting the method of receiving the conversation based on the estimated emotion. The conversation receiving unit estimates the subject's emotion and adjusts the method of receiving the conversation based on the estimated emotion. For example, the conversation receiving unit uses generative AI to estimate the subject's emotion. The conversation receiving unit, for example, receives the conversation in a gentle voice when the subject is nervous, such as, “Please relax and speak.” The conversation receiving unit receives the conversation in a friendly voice when the subject is relaxed, such as, “Let's have a pleasant conversation.” Furthermore, the conversation receiving unit receives the conversation quickly when the subject is in a hurry, such as, “You seem to be in a hurry, so let's keep it brief.” Thus, by adjusting the method of receiving the conversation based on the subject's emotion, the conversation receiving unit enables more appropriate responses. Emotion estimation is realized, for example, by using an emotion engine or generative AI with emotion estimation functions. Generative AI may be a text generative AI (e.g., LLM) or a multimodal generative AI, but is not limited to these examples. Specifically, the conversation receiving unit is composed of multiple computer modules, including a speech recognition module, an image analysis module, a natural language processing engine, a multimodal large language model, an emotion estimation algorithm, and a response adjustment module. The conversation receiving unit first has the speech recognition module acquire the subject's spoken voice data (e.g., 16 kHz/16 bit PCM format, shape=[T] waveform array), perform preprocessing such as spectrogram conversion and noise removal, and extract acoustic features (e.g., F0, MFCC, energy, etc.). The image analysis module takes facial image frames acquired from a camera (shape=[H, W, 3] RGB image) as input and performs face detection and facial expression classification (e.g., nervousness, relaxation, smile, etc.) using neural networks such as CNNs or Vision Transformers. The natural language processing engine tokenizes the spoken text sequence (e.g., “I'm in a bit of a hurry today”) and converts it into context vectors (e.g., shape=[N, 768]). The multimodal large language model integrates acoustic feature vectors, facial expression feature vectors, and spoken text embedding vectors, and outputs emotion labels (e.g., nervousness, relaxation, hurry, etc.) and emotion scores (e.g., nervousness 0.81, relaxation 0.12, etc.). Input examples include: (1) a fast-spoken voice saying “I'm in a bit of a hurry today” plus a serious facial expression image; (2) a calm voice saying “I can talk slowly today” plus a smiling face image; (3) a trembling voice saying “I'm nervous because it's my first time” plus a tense facial expression image. Output examples include: (1) emotion label “hurry,” score 0.88; (2) emotion label “relaxation,” score 0.93; (3) emotion label “nervousness,” score 0.85. The response adjustment module uses these emotion estimation results to automatically adjust parameters for speech synthesis during conversation reception (e.g., voice tone, speaking speed, intonation), reception message content (e.g., “Please relax and speak,” “You seem to be in a hurry, so let's keep it brief,” etc.), and reception interface (e.g., button size, response time limit, etc.). Subsequent processing includes recording emotion estimation results and adjustment details at reception in the conversation history database and using them for future reception optimization and anomaly detection. Internally, the model performs supervised learning and transfer learning using cross-entropy loss and MSE loss to improve emotion classification accuracy, and pre-trains on diverse emotion datasets. As a technical effect, the conversation receiving unit realizes integrated analysis of multidimensional features (speech, facial expression, text) and automatic optimization of reception method according to emotion, which is difficult with human subjective judgment or simple rule-based processing, thereby greatly improving the naturalness of communication, user satisfaction, and psychological care effect. In addition, by utilizing parallel computing with GPUs and distributed inference infrastructure, real-time emotion-adaptive reception adjustment for many subjects can be processed quickly. Specific application fields include elderly monitoring services, remote caregiving support, home medical monitoring, support for people with disabilities, customer support, and mental health care.

The conversation receiving unit is capable of referring to the subject's past conversation history at the time of receiving a conversation and selecting an optimal receiving method. The conversation receiving unit refers to the subject's past conversation history at the time of receiving a conversation and selects an optimal receiving method. For example, the conversation receiving unit prioritizes conversation styles that the subject preferred in the past, such as, “Let's continue the conversation we had previously.” The conversation receiving unit avoids topics that the subject avoided in the past, such as, “We will avoid topics you previously preferred not to discuss.” Furthermore, the conversation receiving unit prioritizes topics that the subject frequently talked about in the past, such as, “Let's talk about topics you often discussed before.” Thus, by referring to the subject's past conversation history, the conversation receiving unit can select a more appropriate receiving method. Specifically, the conversation receiving unit is composed of multiple computer modules, including a conversation history database reference module, a natural language processing engine, a history feature extraction module, a receiving method selection algorithm, and a response generation module. The conversation receiving unit first has the conversation history database reference module acquire conversation history data for the past 30 days (e.g., structured data including date, time, spoken text, summary, emotion label, etc., shape=[30, L]). The natural language processing engine tokenizes each spoken text and converts it into context vectors (e.g., shape=[N, 768]). The history feature extraction module performs topic clustering (e.g., K-means, LDA, etc.) and TF-IDF scoring on these vectors to extract frequently occurring topic categories (e.g., hobbies, health, family, etc.), preferred conversation styles (e.g., casual conversation, Q&A, detailed explanation, etc.), and avoided topics (e.g., health issues, family topics, etc.). The receiving method selection algorithm comprehensively evaluates extracted history features, conversation trends, and emotion transitions to automatically determine the most appropriate conversation style, topic selection, and response template at the time of reception. For example, if the “health” topic was avoided in the past week, the “health”-related topic is excluded and “hobby” or “family” topics are prioritized. The response generation module generates reception messages such as “Let's continue the conversation we had previously,” “We will avoid topics you previously preferred not to discuss,” etc., based on the selected receiving method. Input examples include: (1) conversation history data for the past 30 days (e.g., “I played with my grandchild last week,” “I want to avoid talking about health,” etc.); (2) frequently occurring topic list (e.g., “walking,” “family,” etc.); (3) avoided topic list (e.g., “health,” etc.). Output examples include: (1) “Let's talk about topics you often discussed before”; (2) “We will avoid topics you previously preferred not to discuss.” Subsequent processing includes recording receiving method selection results in the conversation history database and using them for future reception optimization and individual optimization. As a technical effect, the conversation receiving unit realizes high-precision personalized receiving method recommendation based on past history, which is difficult with human subjective memory or simple random reception, by automating high-dimensional feature analysis, clustering, and natural language generation by AI, thereby greatly improving the affinity, continuity, and user satisfaction of communication. In addition, by utilizing parallel computing with GPUs and distributed inference infrastructure, analysis of conversation history and receiving method selection for many subjects can be processed quickly. Specific application fields include elderly monitoring services, remote caregiving support, home medical monitoring, support for people with disabilities, personal assistants, and customer support.

The conversation receiving unit is capable of adjusting the method of receiving a conversation at the time of reception based on the subject's current activity status. The conversation receiving unit adjusts the method of receiving a conversation at the time of reception based on the subject's current activity status. For example, the conversation receiving unit prioritizes short conversations when the subject is exercising, such as, “It seems you are exercising, so let's keep it brief.” The conversation receiving unit accepts longer conversations when the subject is resting, such as, “It seems you are resting, so let's have a leisurely conversation.” Furthermore, the conversation receiving unit prioritizes topics related to meals when the subject is eating, such as, “It seems you are eating, so let's talk about meals.” Thus, by adjusting the method of receiving a conversation based on the subject's current activity status, the conversation receiving unit enables more appropriate responses. Specifically, the conversation receiving unit is composed of multiple computer modules, including an activity status estimation module, a sensor data acquisition module, an image analysis module, an activity classification algorithm, and a reception adjustment module. The conversation receiving unit first has the sensor data acquisition module acquire acceleration data (e.g., shape=[T, 3]), environmental sound data (e.g., shape=[T, 1]), camera images (e.g., shape=[H, W, 3]), etc., in real time from the subject's wearable devices or smart home sensors. The image analysis module uses neural networks such as CNNs or Vision Transformers to estimate activity labels such as “exercise,” “rest,” “meal,” etc., from images. The activity classification algorithm integrates sensor features (e.g., acceleration patterns, acoustic features, image features) to accurately determine the current activity category (e.g., exercising, resting, eating, etc.). The reception adjustment module automatically adjusts the reception interface (e.g., conversation time limit, topic template, response speed, etc.) and reception message content (e.g., “It seems you are exercising, so let's keep it brief,” “It seems you are resting, so let's have a leisurely conversation,” etc.) according to the estimated activity category. Input examples include: (1) exercise pattern from acceleration data plus outdoor image→“exercising” determination; (2) sofa image plus stationary acceleration→“resting” determination; (3) dining table image plus dish sound→“eating” determination. Output examples include: (1) “It seems you are exercising, so let's keep it brief”; (2) “It seems you are resting, so let's have a leisurely conversation”; (3) “It seems you are eating, so let's talk about meals.” Subsequent processing includes recording activity estimation and reception adjustment results in the conversation history database and using them for future individual optimization and anomaly detection. As a technical effect, the conversation receiving unit realizes real-time activity recognition and reception optimization that is difficult with human visual observation or simple schedule-based methods, by automating multidimensional sensor data analysis, image recognition, and natural language generation by AI, thereby greatly improving the immediacy, affinity, and user satisfaction of communication. In addition, by utilizing parallel computing with GPUs and distributed inference infrastructure, activity recognition and reception adjustment for many subjects can be processed quickly. Specific application fields include elderly monitoring services, remote caregiving support, home medical monitoring, support for people with disabilities, personal assistants, and smart homes.

The conversation receiving unit can estimate the emotion of the subject and determine the priority for receiving conversations based on the estimated emotion. The conversation receiving unit estimates the emotion of the subject and determines the priority for receiving conversations based on the estimated emotion. For example, the conversation receiving unit may use a generative AI to estimate the emotion of the subject. If the subject is sad, the conversation receiving unit prioritizes receiving the conversation. For example, the content may be, “I am here to listen, please feel free to talk.” If the subject is happy, the conversation receiving unit receives the conversation with normal priority. For example, the content may be, “I am here to listen, please feel free to talk.” Furthermore, if the subject is tired, the conversation receiving unit prioritizes short conversations. For example, the content may be, “You seem tired, let's keep it brief.” By determining the priority for receiving conversations based on the subject's emotion, the conversation receiving unit can provide more appropriate responses. Emotion estimation is realized by using an emotion estimation function, for example, with an emotion engine or generative AI. The generative AI may be a text generation AI (e.g., LLM) or a multimodal generative AI, but is not limited to these examples. Specifically, the conversation receiving unit is composed of multiple computer modules such as a speech recognition module, image analysis module, natural language processing engine, multimodal large language model, emotion estimation algorithm, and priority determination module. First, the speech recognition module acquires the subject's speech audio data (e.g., 16 kHz/16 bit PCM format, shape=[T] waveform array), performs preprocessing such as spectrogram conversion and noise reduction, and extracts acoustic features (e.g., F0, MFCC, energy, etc.). The image analysis module takes facial image frames obtained from a camera (shape=[H, W, 3] RGB images) as input and performs face detection and facial expression classification (e.g., sadness, happiness, fatigue, etc.) using neural networks such as CNNs or Vision Transformers. The natural language processing engine tokenizes the utterance text sequence (e.g., “I'm a bit tired today”) and converts it into a context vector (e.g., shape=[N, 768]). The multimodal large language model integrates acoustic feature vectors, facial expression feature vectors, and text embedding vectors, and outputs emotion labels (e.g., sadness, happiness, fatigue, etc.) and emotion scores (e.g., sadness 0.81, happiness 0.12, fatigue 0.65, etc.). Input examples include: (1) a depressed voice saying “I'm very sad today”+a sad facial image, (2) a cheerful voice saying “I'm very happy today”+a smiling facial image, (3) a flat voice saying “I've been a bit tired lately”+a neutral facial image, etc. Output examples include: (1) emotion label “sadness”, score 0.91; (2) emotion label “happiness”, score 0.93; (3) emotion label “fatigue”, score 0.78, etc. The priority determination module assigns priority scores to conversation requests in the reception queue based on these emotion estimation results, increases priority when sadness or fatigue is high, sets standard priority for normal or happy states, and prioritizes short conversations for fatigue, automatically adjusting the reception order using rule-based or learning-based algorithms. Subsequent processing reflects the prioritized conversation requests in the reception interface, allowing service users or system administrators to respond in order of priority. Internally, supervised learning and transfer learning using cross-entropy loss or MSE loss are performed to improve emotion classification accuracy, with pre-training on diverse emotion datasets. As a technical effect, the conversation receiving unit achieves integrated analysis of multidimensional features (voice, facial expression, text) and automatic optimization of reception priority according to emotion, which is difficult with human subjective judgment or simple reception order, thereby greatly improving communication urgency, user satisfaction, and psychological care effects. Furthermore, by utilizing parallel computation with GPUs and distributed inference infrastructure, real-time emotion-adaptive reception priority determination for many subjects can be processed at high speed. Specific application fields include elderly monitoring services, remote care support, home medical monitoring, support for people with disabilities, customer support, and mental health care.

The conversation receiving unit can prioritize receiving highly relevant conversations by considering the geographic location information of the subject at the time of conversation reception. The conversation receiving unit prioritizes receiving highly relevant conversations by considering the geographic location information of the subject at the time of conversation reception. For example, if the subject is in a hospital, the conversation receiving unit prioritizes health-related conversations. For example, the content may be, “How was your medical examination at the hospital?” If the subject is in a park, the conversation receiving unit prioritizes nature-related conversations. For example, the content may be, “Is your walk in the park pleasant?” Furthermore, if the subject is in a shopping mall, the conversation receiving unit prioritizes shopping-related conversations. For example, the content may be, “Are you enjoying shopping?” By considering the geographic location information of the subject when receiving conversations, the conversation receiving unit can provide more appropriate responses. Specifically, the conversation receiving unit is composed of multiple computer modules such as a location information acquisition module, geographic context estimation module, conversation relevance evaluation module, priority determination module, and natural language processing engine. First, the location information acquisition module obtains real-time latitude and longitude data (e.g., shape=[2]) from GPS sensors or smartphones. The geographic context estimation module matches the data with a map database or POI (Point of Interest) information to determine whether the current location corresponds to categories such as “hospital,” “park,” or “shopping mall.” The conversation relevance evaluation module extracts conversation categories related to the current location (e.g., health, nature, shopping, etc.) from conversation requests in the reception queue and past conversation history, and calculates a relevance score (e.g., 0.92). The priority determination module places conversation requests with high relevance scores at the top of the reception order. Input examples include: (1) GPS data+POI “hospital”→prioritize health category conversations; (2) GPS data+POI “park”→prioritize nature category conversations; (3) GPS data+POI “shopping mall”→prioritize shopping category conversations, etc. Output examples include: (1) “How was your medical examination at the hospital?”; (2) “Is your walk in the park pleasant?”; (3) “Are you enjoying shopping?” These conversations are prioritized for reception. Subsequent processing reflects the prioritized conversation requests in the reception interface, allowing service users or system administrators to respond to highly relevant conversations in order. As a technical effect, the conversation receiving unit automates real-time location-linked conversation priority optimization, which is difficult with manual human confirmation or simple reception order, by using AI-based geographic context estimation and relevance evaluation, thereby greatly improving immediacy, affinity, and user satisfaction in communication. Furthermore, by utilizing parallel computation with GPUs and distributed inference infrastructure, location-linked conversation reception for many subjects can be processed at high speed. Specific application fields include elderly monitoring services, remote care support, home medical monitoring, support for people with disabilities, personal assistants, and location-linked services.

The conversation receiving unit can analyze the subject's social media activity at the time of conversation reception and receive relevant conversations. The conversation receiving unit analyzes the subject's social media activity at the time of conversation reception and receives relevant conversations. For example, the conversation receiving unit receives conversations related to photos shared by the subject on social media. For example, the content may be, “That's a wonderful photo!” The conversation receiving unit also receives conversations related to comments made by the subject on social media. For example, the content may be, “Please tell me more about that comment.” Furthermore, the conversation receiving unit receives conversations related to accounts followed by the subject on social media. For example, the content may be, “What do you think about that account?” By analyzing the subject's social media activity, the conversation receiving unit can receive more appropriate conversations. Specifically, the conversation receiving unit is composed of multiple computer modules such as a social media data acquisition module, natural language processing engine, image analysis module, topic extraction algorithm, conversation relevance evaluation module, and priority determination module. First, the social media data acquisition module obtains the latest post data (e.g., text posts, images, comments, list of followed accounts, etc.) via API or similar, with the subject's permission. The natural language processing engine tokenizes post texts and comments and converts them into context vectors (e.g., shape=[N, 768]). The image analysis module analyzes posted images (e.g., shape=[H, W, 3]) using CNNs or Vision Transformers to estimate image content (e.g., landscape, food, people, etc.) and emotion labels. The topic extraction algorithm integrates features from post texts, images, comments, and followed accounts to extract the latest interests and topic categories (e.g., travel, hobbies, health, etc.). The conversation relevance evaluation module calculates relevance scores (e.g., 0.89) between conversation requests in the reception queue and extracted topic categories, and the priority determination module places highly relevant conversation requests at the top of the reception order. Input examples include: (1) latest posted image “cherry blossom photo”+text “I went to the park”; (2) comment “I went to a new restaurant”; (3) followed account “culinary expert,” etc. Output examples include: (1) “That's a wonderful photo!”; (2) “Please tell me more about that comment.”; (3) “What do you think about that account?” These conversations are prioritized for reception. Subsequent processing reflects the prioritized conversation requests in the reception interface, allowing service users or system administrators to respond to conversations that match the latest interests in order. As a technical effect, the conversation receiving unit automates real-time, multimodal social media-linked conversation priority optimization, which is difficult with manual human confirmation or simple reception order, by using AI-based natural language and image analysis and feature integration, thereby greatly improving immediacy, affinity, and user satisfaction in communication. Furthermore, by utilizing parallel computation with GPUs and distributed inference infrastructure, social media-linked conversation reception for many subjects can be processed at high speed. Specific application fields include elderly monitoring services, remote care support, home medical monitoring, support for people with disabilities, personal assistants, and customer support.

The summarizing unit can estimate the emotion of the subject and adjust the expression style of the summary based on the estimated emotion. The summarizing unit estimates the emotion of the subject and adjusts the expression style of the summary based on the estimated emotion. For example, the summarizing unit may use a generative AI to estimate the emotion of the subject. If the subject is sad, the summarizing unit generates a summary with gentle expressions. For example, the content may be, “The subject spoke with a slightly sad tone.” If the subject is happy, the summarizing unit generates a summary with bright expressions. For example, the content may be, “The subject spoke with a very happy tone.” Furthermore, if the subject is tired, the summarizing unit generates a concise summary. For example, the content may be, “The subject seemed a bit tired.” By adjusting the expression style of the summary based on the subject's emotion, the summarizing unit can generate more appropriate summaries. Emotion estimation is realized by using an emotion estimation function, for example, with an emotion engine or generative AI. The generative AI may be a text generation AI (e.g., LLM) or a multimodal generative AI, but is not limited to these examples. Specifically, the summarizing unit is composed of multiple computer modules such as a speech recognition module, image analysis module, natural language processing engine, multimodal large language model, emotion estimation algorithm, and summary generation algorithm. First, the speech recognition module acquires the subject's speech audio data (e.g., 16 kHz/16 bit PCM format, shape=[T] waveform array), performs preprocessing such as spectrogram conversion and noise reduction, and extracts acoustic features (e.g., F0, MFCC, energy, etc.). The image analysis module takes facial image frames obtained from a camera (shape=[H, W, 3] RGB images) as input and performs face detection and facial expression classification (e.g., sadness, happiness, fatigue, etc.) using neural networks such as CNNs or Vision Transformers. The natural language processing engine tokenizes the utterance text sequence (e.g., “I'm a bit tired today”) and converts it into a context vector (e.g., shape=[N, 768]). The multimodal large language model integrates acoustic feature vectors, facial expression feature vectors, and text embedding vectors, and outputs emotion labels (e.g., sadness, happiness, fatigue, etc.) and emotion scores (e.g., sadness 0.81, happiness 0.12, fatigue 0.65, etc.). Input examples include: (1) a depressed voice saying “I'm very sad today”+a sad facial image, (2) a cheerful voice saying “I'm very happy today”+a smiling facial image, (3) a flat voice saying “I've been a bit tired lately”+a neutral facial image, etc. Output examples include: (1) emotion label “sadness”, score 0.91; (2) emotion label “happiness”, score 0.93; (3) emotion label “fatigue”, score 0.78, etc. Based on these emotion estimation results, the summarizing unit provides the emotion label as a prompt to the summary generation algorithm (e.g., conditional text generation, emotion-controlled summary generation), and automatically switches the expression style of the summary (e.g., gentle expression, bright expression, concise expression, etc.). For example, for the sadness label, the summary generated is “The subject spoke with a slightly sad tone”; for the happiness label, “The subject spoke with a very happy tone”; for the fatigue label, “The subject seemed a bit tired,” etc. Internally, the model simultaneously optimizes emotion classification accuracy and summary generation accuracy using cross-entropy loss or MSE loss as the loss function, and utilizes supervised learning and transfer learning for pre-training on diverse emotion and summary datasets. Subsequent processing records the generated summary in the storage unit, allowing service users or care staff to view summaries according to emotion. As a technical effect, the summarizing unit achieves integrated analysis of multidimensional features (voice, facial expression, text) and automatic optimization of summary expression according to emotion, which is difficult with human subjective judgment or simple rule-based processing, thereby greatly improving the naturalness of summaries, user satisfaction, and psychological care effects. Furthermore, by utilizing parallel computation with GPUs and distributed inference infrastructure, emotion-adaptive summary generation for many subjects can be processed at high speed. Specific application fields include elderly monitoring services, remote care support, home medical monitoring, support for people with disabilities, mental health care, and customer support.

The summarizing unit can adjust the level of detail of the summary based on the importance of the conversation at the time of summary generation. The summarizing unit adjusts the level of detail of the summary based on the importance of the conversation at the time of summary generation. For example, the summarizing unit may use a generative AI to evaluate the importance of the conversation. If the conversation is important, the summarizing unit generates a detailed summary. For example, the content may be, “The subject talked about an important schedule.” If the conversation is general, the summarizing unit generates a concise summary. For example, the content may be, “The subject talked about daily events.” Furthermore, if the conversation is short, the summarizing unit summarizes only the main points. For example, the content may be, “The subject gave a short greeting.” By adjusting the level of detail of the summary based on the importance of the conversation, the summarizing unit can appropriately summarize important information. Specifically, the summarizing unit is composed of multiple computer modules such as a natural language processing engine, large language model, importance evaluation module, and summary generation algorithm. First, the conversation text sequence (e.g., shape=[L] token sequence) is input to the natural language processing engine, which performs morphological analysis and tokenization. The large language model (e.g., Transformer architecture) generates context vectors (e.g., shape=[N, 768]), calculates the importance score of the conversation (e.g., 0.92) using attention weights, TF-IDF scores, and contextual features. The importance evaluation module comprehensively evaluates keyword frequency in the conversation content, relevance to past conversation history, and user-set priorities, and automatically adjusts the parameters of the summary generation algorithm (e.g., summary length, level of detail, number of extracted sentences, etc.) according to the importance. Input examples include: (1) “I have a hospital appointment tomorrow” (important schedule), (2) “The weather is nice today” (general utterance), (3) “Good morning” (short greeting), etc. Output examples include: (1) “The subject talked about an appointment to go to the hospital tomorrow”; (2) “The subject talked about daily events”; (3) “The subject gave a short greeting,” etc. Subsequent processing records the generated summary in the storage unit, allowing service users or care staff to view summaries according to importance. Internally, the model optimizes summary generation accuracy using supervised learning or reinforcement learning, with importance-weighted cross-entropy loss as the loss function. As a technical effect, the summarizing unit automates context-dependent importance evaluation and dynamic optimization of summary detail, which is difficult with human subjective judgment or simple rule-based processing, by using AI-based high-dimensional feature analysis, thereby greatly improving summary accuracy, information value, and user satisfaction. Furthermore, by utilizing parallel computation with GPUs and distributed inference infrastructure, importance evaluation and summary generation for large volumes of conversation data can be processed at high speed. Specific application fields include elderly monitoring services, remote care support, home medical monitoring, support for people with disabilities, customer support, and automatic FAQ generation.

The summarizing unit can apply different summarization algorithms according to the category of the conversation at the time of summary generation. The summarizing unit applies different summarization algorithms according to the category of the conversation at the time of summary generation. For example, the summarizing unit may use a generative AI to classify the category of the conversation. If the conversation is about health, the summarizing unit generates a detailed summary. For example, the content may be, “The subject talked in detail about their health condition.” If the conversation is about hobbies, the summarizing unit generates a concise summary. For example, the content may be, “The subject briefly talked about their hobbies.” Furthermore, if the conversation is about family, the summarizing unit summarizes the main points. For example, the content may be, “The subject talked about recent family updates.” By applying the optimal summarization algorithm according to the category of the conversation, the summarizing unit improves the accuracy of the summary. Specifically, the summarizing unit is composed of multiple computer modules such as a natural language processing engine, large language model, category classification module, summary algorithm selection module, and summary generation algorithm. First, the conversation text sequence (e.g., shape=[L] token sequence) is input to the natural language processing engine, which performs morphological analysis and tokenization. The large language model generates context vectors (e.g., shape=[N, 768]), and the category classification module classifies the conversation content into categories such as “health,” “hobbies,” or “family.” The summary algorithm selection module automatically selects the optimal summarization algorithm for each category (e.g., extractive summarization, generative summarization, keyword extraction-based summarization, etc.) and provides category information as a prompt to the summary generation algorithm. Input examples include: (1) “My blood pressure has been high recently” (health category), (2) “I enjoy gardening” (hobby category), (3) “I played with my grandchild” (family category), etc. Output examples include: (1) “The subject talked in detail about their health condition”; (2) “The subject briefly talked about their hobbies”; (3) “The subject talked about recent family updates,” etc. Subsequent processing records the category-specific summary results in the storage unit, and displays or analyzes them by category on dashboards or in report generation. Internally, the model improves category classification accuracy using supervised learning or transfer learning with cross-entropy loss, and optimizes in conjunction with summary generation accuracy. As a technical effect, the summarizing unit automates category-dependent summarization algorithm selection and optimization, which is difficult with human subjective judgment or simple rule-based processing, by using AI-based high-dimensional feature analysis and category classification, thereby greatly improving summary accuracy, information value, and user satisfaction. Furthermore, by utilizing parallel computation with GPUs and distributed inference infrastructure, category-specific summary generation for many subjects can be processed at high speed. Specific application fields include elderly monitoring services, remote care support, home medical monitoring, support for people with disabilities, customer support, and automatic FAQ generation.

The summarizing unit can estimate the emotion of the subject and adjust the length of the summary based on the estimated emotion. The summarizing unit estimates the emotion of the subject and adjusts the length of the summary based on the estimated emotion. For example, the summarizing unit may use a generative AI to estimate the emotion of the subject. If the subject is sad, the summarizing unit provides a short summary. For example, the content may be, “The subject spoke with a slightly sad tone.” If the subject is happy, the summarizing unit provides a long summary. For example, the content may be, “The subject spoke with a very happy tone.” Furthermore, if the subject is tired, the summarizing unit provides a concise summary. For example, the content may be, “The subject seemed a bit tired.” By adjusting the length of the summary based on the subject's emotion, the summarizing unit can generate more appropriate summaries. Emotion estimation is realized by using an emotion estimation function, for example, with an emotion engine or generative AI. The generative AI may be a text generation AI (e.g., LLM) or a multimodal generative AI, but is not limited to these examples. Specifically, the summarizing unit is composed of multiple computer modules such as a speech recognition module, image analysis module, natural language processing engine, multimodal large language model, emotion estimation algorithm, and summary length adjustment algorithm. First, the speech recognition module acquires the subject's speech audio data (e.g., 16 kHz/16 bit PCM format, shape=[T] waveform array), performs preprocessing such as spectrogram conversion and noise reduction, and extracts acoustic features (e.g., F0, MFCC, energy, etc.). The image analysis module takes facial image frames obtained from a camera (shape=[H, W, 3] RGB images) as input and performs face detection and facial expression classification (e.g., sadness, happiness, fatigue, etc.) using neural networks such as CNNs or Vision Transformers. The natural language processing engine tokenizes the utterance text sequence (e.g., “I'm a bit tired today”) and converts it into a context vector (e.g., shape=[N, 768]). The multimodal large language model integrates acoustic feature vectors, facial expression feature vectors, and text embedding vectors, and outputs emotion labels (e.g., sadness, happiness, fatigue, etc.) and emotion scores (e.g., sadness 0.81, happiness 0.12, fatigue 0.65, etc.). The summary length adjustment algorithm automatically adjusts the length of the summary (e.g., number of tokens, number of sentences, etc.) according to the emotion label, generating a short summary for sadness, a long summary for happiness, and a concise summary for fatigue. Input examples include: (1) a depressed voice saying “I'm very sad today”+a sad facial image, (2) a cheerful voice saying “I'm very happy today”+a smiling facial image, (3) a flat voice saying “I've been a bit tired lately”+a neutral facial image, etc. Output examples include: (1) “The subject spoke with a slightly sad tone”; (2) “The subject spoke with a very happy tone”; (3) “The subject seemed a bit tired,” etc. Subsequent processing records the generated summary in the storage unit, allowing service users or care staff to view summaries according to emotion and summary length. Internally, the model optimizes summary generation accuracy using emotion label-controlled summary length loss as the loss function, and utilizes supervised learning and transfer learning for pre-training on diverse emotion and summary length datasets. As a technical effect, the summarizing unit automates emotion-dependent summary length control, which is difficult with human subjective judgment or simple rule-based processing, by using AI-based multidimensional feature analysis and natural language generation, thereby greatly improving the naturalness of summaries, user satisfaction, and psychological care effects. Furthermore, by utilizing parallel computation with GPUs and distributed inference infrastructure, emotion-adaptive summary length control for many subjects can be processed at high speed. Specific application fields include elderly monitoring services, remote care support, home medical monitoring, support for people with disabilities, mental health care, and customer support.

The storage unit can determine the priority of summaries based on the submission timing of conversations at the time of summary generation. The storage unit determines the priority of summaries based on the submission timing of conversations at the time of summary generation. For example, the storage unit may use a generative AI to identify the submission timing of conversations. The storage unit prioritizes summarizing the latest conversations. For example, the content may be, “The latest conversation content was prioritized for summarization.” The storage unit postpones summarizing past conversations. For example, the content may be, “Past conversation content was postponed for summarization.” Furthermore, the storage unit prioritizes summarizing conversations from specific time periods. For example, the content may be, “Conversation content from specific time periods was prioritized for summarization.” By determining the priority of summaries based on the submission timing of conversations, the storage unit can prioritize summarizing the latest information. Specifically, the storage unit is composed of multiple computer modules such as a conversation reception timestamp management module, priority determination algorithm, natural language processing engine, and summary generation algorithm. First, the conversation reception timestamp management module records the submission time of each conversation data (e.g., UNIX timestamp, shape=[1]). The priority determination algorithm calculates priority scores (e.g., 0.95, 0.5, 0.8, etc.) for latest conversations, past conversations, and conversations from specific time periods based on submission timing information, and automatically adjusts the order of the summary generation queue. The natural language processing engine tokenizes conversation text sequences in order of priority, and the summary generation algorithm sequentially generates summary sentences. Input examples include: (1) latest conversation “I feel well today”; (2) conversation from one week ago “I played with my grandchild last week”; (3) conversation from a specific time period (e.g., nighttime) “I couldn't sleep at night,” etc. Output examples include: (1) “The latest conversation content was prioritized for summarization”; (2) “Past conversation content was postponed for summarization”; (3) “Conversation content from specific time periods was prioritized for summarization,” etc. Subsequent processing records summary priority information in the storage unit or dashboard, allowing service users to quickly grasp the latest information. Internally, the model optimizes summary generation accuracy according to submission timing using priority control summary generation loss for training. As a technical effect, the storage unit automates submission timing-dependent summary priority optimization, which is difficult with manual management or simple FIFO processing, by using AI-based timestamp management and priority control, thereby greatly improving information freshness, response speed, and user satisfaction. Furthermore, by utilizing parallel computation with GPUs and distributed inference infrastructure, priority-based summary generation for large volumes of conversation data can be processed at high speed. Specific application fields include elderly monitoring services, remote care support, home medical monitoring, support for people with disabilities, customer support, and automatic FAQ generation.

The summarizing unit can adjust the order of summaries based on the relevance of conversations at the time of summary generation. The summarizing unit adjusts the order of summaries based on the relevance of conversations at the time of summary generation. For example, the summarizing unit may use a generative AI to evaluate the relevance of conversations. The summarizing unit summarizes important conversations first. For example, the content may be, “Important conversation content was summarized first.” The summarizing unit postpones general conversations. For example, the content may be, “General conversation content was postponed for summarization.” Furthermore, the summarizing unit prioritizes summarizing highly relevant conversations. For example, the content may be, “Highly relevant conversation content was prioritized for summarization.” By adjusting the order of summaries based on the relevance of conversations, the summarizing unit can prioritize summarizing important information. Specifically, the summarizing unit is composed of multiple computer modules such as a natural language processing engine, large language model, relevance evaluation module, summary order control algorithm, and summary generation algorithm. First, the conversation text sequence (e.g., shape=[L] token sequence) is input to the natural language processing engine, which generates context vectors (e.g., shape=[N, 768]). The relevance evaluation module comprehensively evaluates keyword frequency in the conversation content, similarity to past conversation history, and user-set priority categories, and calculates relevance scores (e.g., 0.92, 0.75, 0.61, etc.). The summary order control algorithm automatically adjusts the order of the summary generation queue based on relevance scores, prioritizing important and highly relevant conversations for summarization. The summary generation algorithm generates summary sentences based on the order-controlled conversation data. Input examples include: (1) “I have a hospital appointment tomorrow” (important conversation), (2) “The weather is nice today” (general conversation), (3) “I played with my grandchild” (highly relevant conversation), etc. Output examples include: (1) “Important conversation content was summarized first”; (2) “General conversation content was postponed for summarization”; (3) “Highly relevant conversation content was prioritized for summarization,” etc. Subsequent processing records summary order information in the storage unit or dashboard, allowing service users to quickly grasp important information. Internally, the model optimizes summary generation accuracy according to relevance using relevance-weighted summary generation loss for training. As a technical effect, the summarizing unit automates relevance-dependent summary order optimization, which is difficult with human subjective judgment or simple rule-based processing, by using AI-based high-dimensional feature analysis and relevance evaluation, thereby greatly improving information value, response speed, and user satisfaction. Furthermore, by utilizing parallel computation with GPUs and distributed inference infrastructure, relevance-based summary generation for large volumes of conversation data can be processed at high speed. Specific application fields include elderly monitoring services, remote care support, home medical monitoring, support for people with disabilities, customer support, and automatic FAQ generation.

The storage unit can estimate the emotion of the subject and select the data to be stored based on the estimated emotion. The storage unit estimates the emotion of the subject and selects the data to be stored based on the estimated emotion. For example, the storage unit may use a generative AI to estimate the emotion of the subject. If the subject is sad, the storage unit prioritizes storing important conversations. For example, the content may be, “Because the subject is sad, important conversation content was prioritized for storage.” If the subject is happy, the storage unit stores all conversations. For example, the content may be, “Because the subject is happy, all conversation content was stored.” Furthermore, if the subject is tired, the storage unit prioritizes storing short conversations. For example, the content may be, “Because the subject is tired, short conversation content was prioritized for storage.” By selecting the data to be stored based on the subject's emotion, the storage unit can appropriately store important information. Emotion estimation is realized by using an emotion estimation function, for example, with an emotion engine or generative AI. The generative AI may be a text generation AI (e.g., LLM) or a multimodal generative AI, but is not limited to these examples.

The storage unit can refer to past stored data at the time of storage to select the optimal storage method. The storage unit refers to past stored data at the time of storage to select the optimal storage method. For example, the storage unit stores past stored data in a database and compares it with the current storage method. The storage unit analyzes past stored data to select the optimal storage format. For example, the content may be, “Past stored data was analyzed and the optimal storage format was selected.” The storage unit also refers to past stored data to avoid duplication. For example, the content may be, “Past stored data was referred to and duplication was avoided.” Furthermore, the storage unit determines the priority of storage based on past stored data. For example, the content may be, “The priority of storage was determined based on past stored data.” By referring to past stored data, the storage unit can select the optimal storage method.

The storage unit can determine the priority of storage based on the importance of the data at the time of storage. The storage unit determines the priority of storage based on the importance of the data at the time of storage. For example, the storage unit may use a generative AI to evaluate the importance of the data. The storage unit prioritizes storing important data. For example, the content may be, “Important data was prioritized for storage.” The storage unit postpones storing general data. For example, the content may be, “General data was postponed for storage.” Furthermore, the storage unit prioritizes storing short data. For example, the content may be, “Short data was prioritized for storage.” By determining the priority of storage based on the importance of the data, the storage unit can prioritize storing important information.

The storage unit can estimate the emotion of the subject and adjust the format of the data to be stored based on the estimated emotion. The storage unit estimates the emotion of the subject and adjusts the format of the data to be stored based on the estimated emotion. For example, the storage unit may use a generative AI to estimate the emotion of the subject. If the subject is sad, the storage unit stores the data in a concise format. For example, the content may be, “Because the subject is sad, the data was stored in a concise format.” If the subject is happy, the storage unit stores the data in a detailed format. For example, the content may be, “Because the subject is happy, the data was stored in a detailed format.” Furthermore, if the subject is tired, the storage unit stores the data in a short format. For example, the content may be, “Because the subject is tired, the data was stored in a short format.” By adjusting the format of the data to be stored based on the subject's emotion, the storage unit can store data in a more appropriate format. Emotion estimation is realized by using an emotion estimation function, for example, with an emotion engine or generative AI. The generative AI may be a text generation AI (e.g., LLM) or a multimodal generative AI, but is not limited to these examples.

The storage unit can determine the priority of storage based on the submission timing of the data at the time of storage. The storage unit determines the priority of storage based on the submission timing of the data at the time of storage. For example, the storage unit may use a generative AI to identify the submission timing of the data. The storage unit prioritizes storing the latest data. For example, the content may be, “The latest data was prioritized for storage.” The storage unit postpones storing past data. For example, the content may be, “Past data was postponed for storage.” Furthermore, the storage unit prioritizes storing data from specific time periods. For example, the content may be, “Data from specific time periods was prioritized for storage.” By determining the priority of storage based on the submission timing of the data, the storage unit can prioritize storing the latest information.

The storage unit can adjust the order of storage based on the relevance of the data at the time of storage. The storage unit adjusts the order of storage based on the relevance of the data at the time of storage. For example, the storage unit may use a generative AI to evaluate the relevance of the data. The storage unit stores important data first. For example, the content may be, “Important data was stored first.” The storage unit postpones storing general data. For example, the content may be, “General data was postponed for storage.” Furthermore, the storage unit prioritizes storing highly relevant data. For example, the content may be, “Highly relevant data was prioritized for storage.” By adjusting the order of storage based on the relevance of the data, the storage unit can prioritize storing important information.

The confirmation unit can estimate the emotion of the subject and select the data to be confirmed based on the estimated emotion. The confirmation unit estimates the emotion of the subject and selects the data to be confirmed based on the estimated emotion. For example, the confirmation unit may use a generative AI to estimate the emotion of the subject. If the subject is sad, the confirmation unit prioritizes confirming important data. For example, the content may be, “Because the subject is sad, important data was prioritized for confirmation.” If the subject is happy, the confirmation unit confirms all data. For example, the content may be, “Because the subject is happy, all data was confirmed.” Furthermore, if the subject is tired, the confirmation unit prioritizes confirming short data. For example, the content may be, “Because the subject is tired, short data was prioritized for confirmation.” By selecting the data to be confirmed based on the subject's emotion, the confirmation unit can appropriately confirm important information. Emotion estimation is realized by using an emotion estimation function, for example, with an emotion engine or generative AI. The generative AI may be a text generation AI (e.g., LLM) or a multimodal generative AI, but is not limited to these examples.

The confirmation unit can refer to past confirmation data at the time of confirmation to select the optimal confirmation method. The confirmation unit refers to past confirmation data at the time of confirmation to select the optimal confirmation method. For example, the confirmation unit stores past confirmation data in a database and compares it with the current confirmation method. The confirmation unit analyzes past confirmation data to select the optimal confirmation format. For example, the content may be, “Past confirmation data was analyzed and the optimal confirmation format was selected.” The confirmation unit also refers to past confirmation data to avoid duplication. For example, the content may be, “Past confirmation data was referred to and duplication was avoided.” Furthermore, the confirmation unit determines the priority of confirmation based on past confirmation data. For example, the content may be, “The priority of confirmation was determined based on past confirmation data.” By referring to past confirmation data, the confirmation unit can select the optimal confirmation method.

The confirmation unit can determine the priority of confirmation based on the importance of the data at the time of confirmation. The confirmation unit determines the priority of confirmation based on the importance of the data at the time of confirmation. For example, the confirmation unit may use a generative AI to evaluate the importance of the data. The confirmation unit prioritizes confirming important data. For example, the content may be, “Important data was prioritized for confirmation.” The confirmation unit postpones confirming general data. For example, the content may be, “General data was postponed for confirmation.” Furthermore, the confirmation unit prioritizes confirming short data. For example, the content may be, “Short data was prioritized for confirmation.” By determining the priority of confirmation based on the importance of the data, the confirmation unit can prioritize confirming important information.

The confirmation unit can estimate the emotion of the subject and adjust the format of the data to be confirmed based on the estimated emotion. The confirmation unit estimates the emotion of the subject and adjusts the format of the data to be confirmed based on the estimated emotion. For example, the confirmation unit may use a generative AI to estimate the emotion of the subject. If the subject is sad, the confirmation unit confirms the data in a concise format. For example, the content may be, “Because the subject is sad, the data was confirmed in a concise format.” If the subject is happy, the confirmation unit confirms the data in a detailed format. For example, the content may be, “Because the subject is happy, the data was confirmed in a detailed format.” Furthermore, if the subject is tired, the confirmation unit confirms the data in a short format. For example, the content may be, “Because the subject is tired, the data was confirmed in a short format.” By adjusting the format of the data to be confirmed based on the subject's emotion, the confirmation unit can confirm data in a more appropriate format. Emotion estimation is realized by using an emotion estimation function, for example, with an emotion engine or generative AI. The generative AI may be a text generation AI (e.g., LLM) or a multimodal generative AI, but is not limited to these examples.

The confirmation unit can determine the priority of confirmation based on the submission timing of the data at the time of confirmation. The confirmation unit determines the priority of confirmation based on the submission timing of the data at the time of confirmation. For example, the confirmation unit may use a generative AI to identify the submission timing of the data. The confirmation unit prioritizes confirming the latest data. For example, the content may be, “The latest data was prioritized for confirmation.” The confirmation unit postpones confirming past data. For example, the content may be, “Past data was postponed for confirmation.” Furthermore, the confirmation unit prioritizes confirming data from specific time periods. For example, the content may be, “Data from specific time periods was prioritized for confirmation.” By determining the priority of confirmation based on the submission timing of the data, the confirmation unit can prioritize confirming the latest information.

The confirmation unit can adjust the order of confirmation based on the relevance of the data at the time of confirmation. The confirmation unit adjusts the order of confirmation based on the relevance of the data at the time of confirmation. For example, the confirmation unit evaluates the relevance of the data using a generative AI. The confirmation unit, for example, confirms important data first. For instance, the content may be “Confirmed important data first.” In addition, the confirmation unit postpones general data. For example, the content may be “Postponed general data.” Furthermore, the confirmation unit preferentially confirms highly relevant data. For example, the content may be “Confirmed highly relevant data preferentially.” Thus, by adjusting the order of confirmation based on the relevance of the data, the confirmation unit can preferentially confirm important information.

The emotion analysis unit can estimate the subject's emotion from the content of the conversation and adjust the method of emotion analysis based on the estimated emotion. The emotion analysis unit estimates the subject's emotion from the content of the conversation and adjusts the method of emotion analysis based on the estimated emotion. For example, the emotion analysis unit analyzes the content of the conversation using a generative AI and estimates the subject's emotion. The emotion analysis unit, for example, performs detailed emotion analysis when the subject is sad. For instance, the content may be “Performed detailed emotion analysis because the subject was sad.” In addition, the emotion analysis unit performs concise emotion analysis when the subject is happy. For example, the content may be “Performed concise emotion analysis because the subject was happy.” Furthermore, the emotion analysis unit performs simple emotion analysis when the subject is tired. For example, the content may be “Performed simple emotion analysis because the subject was tired.” Thus, by estimating the subject's emotion from the content of the conversation and adjusting the method of emotion analysis, the emotion analysis unit can perform more appropriate emotion analysis. The estimation of emotion is realized, for example, by using an emotion estimation function such as an emotion engine or generative AI. The generative AI may be a text generative AI (e.g., LLM) or a multimodal generative AI, but is not limited to these examples.

The emotion analysis unit can refer to past emotion data at the time of emotion analysis and select an optimal analysis method. The emotion analysis unit refers to past emotion data at the time of emotion analysis and selects an optimal analysis method. For example, the emotion analysis unit stores past emotion data in a database and compares it with the current analysis method. The emotion analysis unit, for example, analyzes past emotion data and selects an optimal analysis algorithm. For instance, the content may be “Analyzed past emotion data and selected an optimal analysis algorithm.” In addition, the emotion analysis unit refers to past emotion data to avoid duplication. For example, the content may be “Referred to past emotion data and avoided duplication.” Furthermore, the emotion analysis unit determines the priority of analysis based on past emotion data. For example, the content may be “Determined the priority of analysis based on past emotion data.” Thus, by referring to past emotion data, the emotion analysis unit can select an optimal analysis method.

The emotion analysis unit can apply different analysis algorithms according to the category of the conversation at the time of emotion analysis. The emotion analysis unit applies different analysis algorithms according to the category of the conversation at the time of emotion analysis. For example, the emotion analysis unit classifies the category of the conversation using a generative AI. The emotion analysis unit, for example, performs detailed analysis for conversations related to health. For instance, the content may be “The subject talked in detail about their health condition.” In addition, the emotion analysis unit performs concise analysis for conversations related to hobbies. For example, the content may be “The subject talked briefly about their hobbies.” Furthermore, the emotion analysis unit performs focused analysis for conversations related to family. For example, the content may be “The subject talked about the recent status of their family.” Thus, by applying optimal analysis algorithms according to the category of the conversation, the emotion analysis unit can improve the accuracy of emotion analysis.

The emotion analysis unit can estimate the subject's emotion from the content of the conversation and adjust the method of displaying the analysis result based on the estimated emotion. The emotion analysis unit estimates the subject's emotion from the content of the conversation and adjusts the method of displaying the analysis result based on the estimated emotion. For example, the emotion analysis unit analyzes the content of the conversation using a generative AI and estimates the subject's emotion. The emotion analysis unit, for example, displays the analysis result in gentle expressions when the subject is sad. For instance, the content may be “Displayed the analysis result in gentle expressions because the subject was sad.” In addition, the emotion analysis unit displays the analysis result in bright expressions when the subject is happy. For example, the content may be “Displayed the analysis result in bright expressions because the subject was happy.” Furthermore, the emotion analysis unit displays the analysis result in concise expressions when the subject is tired. For example, the content may be “Displayed the analysis result in concise expressions because the subject was tired.” Thus, by estimating the subject's emotion from the content of the conversation and adjusting the method of displaying the analysis result, the emotion analysis unit can provide more appropriate display. The estimation of emotion is realized, for example, by using an emotion estimation function such as an emotion engine or generative AI. The generative AI may be a text generative AI (e.g., LLM) or a multimodal generative AI, but is not limited to these examples.

The emotion analysis unit can determine the priority of analysis based on the submission timing of the conversation at the time of emotion analysis. The emotion analysis unit determines the priority of analysis based on the submission timing of the conversation at the time of emotion analysis. For example, the emotion analysis unit identifies the submission timing of the conversation using a generative AI. The emotion analysis unit, for example, preferentially analyzes the latest conversation. For instance, the content may be “Preferentially analyzed the latest conversation.” In addition, the emotion analysis unit postpones past conversations. For example, the content may be “Postponed past conversations.” Furthermore, the emotion analysis unit preferentially analyzes conversations from specific time periods. For example, the content may be “Preferentially analyzed conversations from specific time periods.” Thus, by determining the priority of analysis based on the submission timing of the conversation, the emotion analysis unit can preferentially analyze the latest information.

The emotion analysis unit can adjust the order of analysis based on the relevance of the conversation at the time of emotion analysis. The emotion analysis unit adjusts the order of analysis based on the relevance of the conversation at the time of emotion analysis. For example, the emotion analysis unit evaluates the relevance of the conversation using a generative AI. The emotion analysis unit, for example, analyzes important conversations first. For instance, the content may be “Analyzed important conversations first.” In addition, the emotion analysis unit postpones general conversations. For example, the content may be “Postponed general conversations.” Furthermore, the emotion analysis unit preferentially analyzes highly relevant conversations. For example, the content may be “Preferentially analyzed highly relevant conversations.” Thus, by adjusting the order of analysis based on the relevance of the conversation, the emotion analysis unit can preferentially analyze important information.

The anomaly detection unit can detect anomalies by comparing with past conversation history and adjust the method of anomaly detection based on the detected anomalies. The anomaly detection unit detects anomalies by comparing with past conversation history and adjusts the method of anomaly detection based on the detected anomalies. For example, the anomaly detection unit analyzes past conversation history using a generative AI and compares it with the current conversation content. The anomaly detection unit, for example, detects abnormal patterns by comparing with past conversation history. For instance, the content may be “Detected abnormal patterns by comparing with past conversation history.” In addition, the anomaly detection unit refers to past conversation history to identify abnormal content. For example, the content may be “Referred to past conversation history and identified abnormal content.” Furthermore, the anomaly detection unit determines the priority of anomaly detection based on past conversation history. For example, the content may be “Determined the priority of anomaly detection based on past conversation history.” Thus, by detecting anomalies by comparing with past conversation history and adjusting the method of anomaly detection, the anomaly detection unit can perform more appropriate anomaly detection.

The anomaly detection unit can refer to past anomaly data at the time of anomaly detection and select an optimal detection method. The anomaly detection unit refers to past anomaly data at the time of anomaly detection and selects an optimal detection method. For example, the anomaly detection unit stores past anomaly data in a database and compares it with the current detection method. The anomaly detection unit, for example, analyzes past anomaly data and selects an optimal detection algorithm. For instance, the content may be “Analyzed past anomaly data and selected an optimal detection algorithm.” In addition, the anomaly detection unit refers to past anomaly data to avoid duplication. For example, the content may be “Referred to past anomaly data and avoided duplication.” Furthermore, the anomaly detection unit determines the priority of detection based on past anomaly data. For example, the content may be “Determined the priority of detection based on past anomaly data.” Thus, by referring to past anomaly data, the anomaly detection unit can select an optimal detection method.

The anomaly detection unit can apply different detection algorithms according to the category of the conversation at the time of anomaly detection. The anomaly detection unit applies different detection algorithms according to the category of the conversation at the time of anomaly detection. For example, the anomaly detection unit classifies the category of the conversation using a generative AI. The anomaly detection unit, for example, performs detailed detection for conversations related to health. For instance, the content may be “The subject talked in detail about their health condition.” In addition, the anomaly detection unit performs concise detection for conversations related to hobbies. For example, the content may be “The subject talked briefly about their hobbies.” Furthermore, the anomaly detection unit performs focused detection for conversations related to family. For example, the content may be “The subject talked about the recent status of their family.” Thus, by applying optimal detection algorithms according to the category of the conversation, the anomaly detection unit can improve the accuracy of anomaly detection.

The anomaly detection unit can detect anomalies by comparing with past conversation history and adjust the method of displaying the detection result based on the detected anomalies. The anomaly detection unit detects anomalies by comparing with past conversation history and adjusts the method of displaying the detection result based on the detected anomalies. For example, the anomaly detection unit analyzes past conversation history using a generative AI and compares it with the current conversation content. The anomaly detection unit, for example, provides a detailed display method when abnormal patterns are detected. For instance, the content may be “Provided a detailed display method because abnormal patterns were detected.” In addition, the anomaly detection unit provides a concise display method when abnormal content is identified. For example, the content may be “Provided a concise display method because abnormal content was identified.” Furthermore, the anomaly detection unit adjusts the display method based on the priority of anomaly detection. For example, the content may be “Adjusted the display method based on the priority of anomaly detection.” Thus, by detecting anomalies by comparing with past conversation history and adjusting the method of displaying the detection result, the anomaly detection unit can provide more appropriate display.

The anomaly detection unit can determine the priority of detection based on the submission timing of the conversation at the time of anomaly detection. The anomaly detection unit determines the priority of detection based on the submission timing of the conversation at the time of anomaly detection. For example, the anomaly detection unit identifies the submission timing of the conversation using a generative AI. The anomaly detection unit, for example, preferentially detects the latest conversation. For instance, the content may be “Preferentially detected the latest conversation.” In addition, the anomaly detection unit postpones past conversations. For example, the content may be “Postponed past conversations.” Furthermore, the anomaly detection unit preferentially detects conversations from specific time periods. For example, the content may be “Preferentially detected conversations from specific time periods.” Thus, by determining the priority of detection based on the submission timing of the conversation, the anomaly detection unit can preferentially detect the latest information.

The anomaly detection unit can adjust the order of detection based on the relevance of the conversation at the time of anomaly detection. The anomaly detection unit adjusts the order of detection based on the relevance of the conversation at the time of anomaly detection. For example, the anomaly detection unit evaluates the relevance of the conversation using a generative AI. The anomaly detection unit, for example, detects important conversations first. For instance, the content may be “Detected important conversations first.” In addition, the anomaly detection unit postpones general conversations. For example, the content may be “Postponed general conversations.” Furthermore, the anomaly detection unit preferentially detects highly relevant conversations. For example, the content may be “Preferentially detected highly relevant conversations.” Thus, by adjusting the order of detection based on the relevance of the conversation, the anomaly detection unit can preferentially detect important information.

The behavior monitoring unit can monitor the behavior of the subject and adjust the monitoring method based on the monitored behavior. The behavior monitoring unit monitors the behavior of the subject and adjusts the monitoring method based on the monitored behavior. For example, the behavior monitoring unit analyzes the behavior of the subject using a generative AI. The behavior monitoring unit, for example, issues an alert when the subject does not move for a certain period of time. For instance, the content may be “Issued an alert because the subject did not move for more than 30 minutes.” In addition, the behavior monitoring unit performs detailed monitoring when the subject exhibits abnormal behavior. For example, the content may be “Performed detailed monitoring because the subject exhibited abnormal behavior.” Furthermore, the behavior monitoring unit determines the priority of monitoring based on the behavior pattern of the subject. For example, the content may be “Determined the priority of monitoring based on the behavior pattern of the subject.” Thus, by monitoring the behavior of the subject and adjusting the monitoring method, the behavior monitoring unit can perform more appropriate monitoring.

The behavior monitoring unit can refer to past behavior data at the time of behavior monitoring and select an optimal monitoring method. The behavior monitoring unit refers to past behavior data at the time of behavior monitoring and selects an optimal monitoring method. For example, the behavior monitoring unit stores past behavior data in a database and compares it with the current monitoring method. The behavior monitoring unit, for example, analyzes past behavior data and selects an optimal monitoring algorithm. For instance, the content may be “Analyzed past behavior data and selected an optimal monitoring algorithm.” In addition, the behavior monitoring unit refers to past behavior data to avoid duplication. For example, the content may be “Referred to past behavior data and avoided duplication.” Furthermore, the behavior monitoring unit determines the priority of monitoring based on past behavior data. For example, the content may be “Determined the priority of monitoring based on past behavior data.” Thus, by referring to past behavior data, the behavior monitoring unit can select an optimal monitoring method.

The behavior monitoring unit can apply different monitoring algorithms according to the category of the behavior at the time of behavior monitoring. The behavior monitoring unit applies different monitoring algorithms according to the category of the behavior at the time of behavior monitoring. For example, the behavior monitoring unit classifies the category of the behavior using a generative AI. The behavior monitoring unit, for example, performs detailed monitoring for behaviors related to health. For instance, the content may be “The subject talked in detail about their health condition.” In addition, the behavior monitoring unit performs concise monitoring for behaviors related to hobbies. For example, the content may be “The subject talked briefly about their hobbies.” Furthermore, the behavior monitoring unit performs focused monitoring for behaviors related to family. For example, the content may be “The subject talked about the recent status of their family.” Thus, by applying optimal monitoring algorithms according to the category of the behavior, the behavior monitoring unit can improve the accuracy of monitoring.

The behavior monitoring unit can monitor the behavior of the subject and adjust the method of displaying the monitoring result based on the monitored behavior. The behavior monitoring unit monitors the behavior of the subject and adjusts the method of displaying the monitoring result based on the monitored behavior. For example, the behavior monitoring unit analyzes the behavior of the subject using a generative AI. The behavior monitoring unit, for example, provides a detailed display method when abnormal behavior is detected. For instance, the content may be “Provided a detailed display method because abnormal behavior was detected.” In addition, the behavior monitoring unit provides a concise display method when abnormal behavior is identified. For example, the content may be “Provided a concise display method because abnormal behavior was identified.” Furthermore, the behavior monitoring unit adjusts the display method based on the priority of monitoring. For example, the content may be “Adjusted the display method based on the priority of monitoring.” Thus, by monitoring the behavior of the subject and adjusting the method of displaying the monitoring result, the behavior monitoring unit can provide more appropriate display.

The behavior monitoring unit can determine the priority of monitoring based on the submission timing of the behavior at the time of behavior monitoring. The behavior monitoring unit determines the priority of monitoring based on the submission timing of the behavior at the time of behavior monitoring. For example, the behavior monitoring unit identifies the submission timing of the behavior using a generative AI. The behavior monitoring unit, for example, preferentially monitors the latest behavior. For instance, the content may be “Preferentially monitored the latest behavior.” In addition, the behavior monitoring unit postpones past behaviors. For example, the content may be “Postponed past behaviors.” Furthermore, the behavior monitoring unit preferentially monitors behaviors from specific time periods. For example, the content may be “Preferentially monitored behaviors from specific time periods.” Thus, by determining the priority of monitoring based on the submission timing of the behavior, the behavior monitoring unit can preferentially monitor the latest information.

The behavior monitoring unit can adjust the order of monitoring based on the relevance of the behavior at the time of behavior monitoring. The behavior monitoring unit adjusts the order of monitoring based on the relevance of the behavior at the time of behavior monitoring. For example, the behavior monitoring unit evaluates the relevance of the behavior using a generative AI. The behavior monitoring unit, for example, monitors important behaviors first. For instance, the content may be “Monitored important behaviors first.” In addition, the behavior monitoring unit postpones general behaviors. For example, the content may be “Postponed general behaviors.” Furthermore, the behavior monitoring unit preferentially monitors highly relevant behaviors. For example, the content may be “Preferentially monitored highly relevant behaviors.” Thus, by adjusting the order of monitoring based on the relevance of the behavior, the behavior monitoring unit can preferentially monitor important information.

The keyword extraction unit can extract important keywords from the content of the conversation and adjust the extraction method based on the extracted keywords. The keyword extraction unit extracts important keywords from the content of the conversation and adjusts the extraction method based on the extracted keywords. For example, the keyword extraction unit analyzes the content of the conversation using a generative AI and extracts important keywords. The keyword extraction unit, for example, extracts important keywords from the content of the conversation and provides a detailed extraction method. For instance, the content may be “Extracted important keywords from the content of the conversation and provided a detailed extraction method.” In addition, the keyword extraction unit extracts important keywords from the content of the conversation and provides a concise extraction method. For example, the content may be “Extracted important keywords from the content of the conversation and provided a concise extraction method.” Furthermore, the keyword extraction unit extracts important keywords from the content of the conversation and determines the priority of extraction. For example, the content may be “Extracted important keywords from the content of the conversation and determined the priority of extraction.” Thus, by extracting important keywords from the content of the conversation and adjusting the extraction method, the keyword extraction unit can perform more appropriate keyword extraction.

The keyword extraction unit can select an optimal extraction method by referring to past keyword data during keyword extraction. The keyword extraction unit selects an optimal extraction method by referring to past keyword data during keyword extraction. For example, the keyword extraction unit stores past keyword data in a database and compares it with the current extraction method. The keyword extraction unit may analyze past keyword data and select an optimal extraction algorithm. For example, the content may be such as “Analyzed past keyword data and selected the optimal extraction algorithm.” In addition, the keyword extraction unit refers to past keyword data to avoid duplication. For example, the content may be such as “Referred to past keyword data and avoided duplication.” Furthermore, the keyword extraction unit determines the priority of extraction based on past keyword data. For example, the content may be such as “Determined the priority of extraction based on past keyword data.” Thus, the keyword extraction unit can select an optimal extraction method by referring to past keyword data.

The keyword extraction unit can apply different extraction algorithms according to the category of the conversation during keyword extraction. The keyword extraction unit applies different extraction algorithms according to the category of the conversation during keyword extraction. For example, the keyword extraction unit classifies the category of the conversation using generative AI. The keyword extraction unit, for example, extracts keywords in detail for conversations related to health. For example, the content may be such as “The subject talked in detail about their health condition.” In addition, the keyword extraction unit extracts keywords concisely for conversations related to hobbies. For example, the content may be such as “The subject talked briefly about their hobbies.” Furthermore, the keyword extraction unit extracts keywords focusing on the main points for conversations related to family. For example, the content may be such as “The subject talked about recent family updates.” Thus, by applying optimal extraction algorithms according to the category of the conversation, the keyword extraction unit can improve the accuracy of keyword extraction.

The keyword extraction unit can extract important keywords from the content of the conversation and adjust the display method based on the extracted keywords. The keyword extraction unit extracts important keywords from the content of the conversation and adjusts the display method based on the extracted keywords. For example, the keyword extraction unit analyzes the content of the conversation using generative AI and extracts important keywords. The keyword extraction unit, for example, displays important keywords in an emphasized manner. For example, the content may be such as “Displayed important keywords in an emphasized manner.” In addition, the keyword extraction unit displays general keywords concisely. For example, the content may be such as “Displayed general keywords concisely.” Furthermore, the keyword extraction unit adjusts the display method based on the priority of extraction. For example, the content may be such as “Adjusted the display method based on the priority of extraction.” Thus, by extracting important keywords from the content of the conversation and adjusting the display method, the keyword extraction unit can provide more appropriate displays.

The keyword extraction unit can determine the priority of extraction based on the submission timing of the conversation during keyword extraction. The keyword extraction unit determines the priority of extraction based on the submission timing of the conversation during keyword extraction. For example, the keyword extraction unit identifies the submission timing of the conversation using generative AI. The keyword extraction unit, for example, preferentially extracts keywords from the latest conversations. For example, the content may be such as “Preferentially extracted keywords from the latest conversations.” In addition, the keyword extraction unit postpones extraction from past conversations. For example, the content may be such as “Postponed extraction from past conversations.” Furthermore, the keyword extraction unit preferentially extracts keywords from conversations in specific time periods. For example, the content may be such as “Preferentially extracted keywords from conversations in specific time periods.” Thus, by determining the priority of extraction based on the submission timing of the conversation, the keyword extraction unit can preferentially extract the latest information.

The keyword extraction unit can adjust the order of extraction based on the relevance of the conversation during keyword extraction. The keyword extraction unit adjusts the order of extraction based on the relevance of the conversation during keyword extraction. For example, the keyword extraction unit evaluates the relevance of the conversation using generative AI. The keyword extraction unit, for example, first extracts keywords from important conversations. For example, the content may be such as “First extracted keywords from important conversations.” In addition, the keyword extraction unit postpones extraction from general conversations. For example, the content may be such as “Postponed extraction from general conversations.” Furthermore, the keyword extraction unit preferentially extracts keywords from highly relevant conversations. For example, the content may be such as “Preferentially extracted keywords from highly relevant conversations.” Thus, by adjusting the order of extraction based on the relevance of the conversation, the keyword extraction unit can preferentially extract important information.

The system according to the embodiment is not limited to the examples described above and, for example, various modifications can be made as follows.

The monitoring system may comprise a health monitoring unit configured to monitor the health status of the subject. The health monitoring unit periodically measures vital signs such as the subject's heart rate and blood pressure, and can issue an alert when an abnormality is detected. For example, an alert is issued when the heart rate is higher than usual or when the blood pressure fluctuates rapidly. In addition, the health monitoring unit may store the measurement results in the storage unit so that the service user can check them. Thus, the health status of the subject can be grasped in real time, and prompt action can be taken when an abnormality occurs.

The monitoring system may comprise a music providing unit configured to estimate the subject's emotion and provide music based on the estimated emotion. The music providing unit estimates the subject's emotion and, for example, provides relaxing music when the subject is feeling sad. When the subject is feeling happy, bright music that further enhances the mood is provided. In addition, when the subject is feeling stressed, music that helps reduce stress can be provided. Thus, by providing music according to the subject's emotion, the system can improve the subject's mood and support mental health.

The monitoring system may comprise a meal recording unit configured to record the subject's meal content. The meal recording unit records the content of the subject's meals and can analyze the nutritional balance. For example, the types and amounts of food consumed by the subject are recorded to grasp the intake status of nutrients. In addition, when the nutritional balance is biased, the meal recording unit can provide advice for improvement. Thus, the system can support the subject's dietary habits and contribute to maintaining health.

The monitoring system may comprise a sleep monitoring unit configured to monitor the subject's sleep status. The sleep monitoring unit measures the subject's sleep time and sleep quality, and can issue an alert when an abnormality is detected. For example, an alert is issued when the sleep time is too short or when the sleep quality deteriorates. In addition, the sleep monitoring unit may store the measurement results in the storage unit so that the service user can check them. Thus, the sleep status of the subject can be grasped in real time, and prompt action can be taken when an abnormality occurs.

The monitoring system may comprise a relaxation proposal unit configured to estimate the subject's emotion and propose relaxation methods based on the estimated emotion. The relaxation proposal unit estimates the subject's emotion and, for example, proposes relaxation methods such as deep breathing or meditation when the subject is feeling stressed. When the subject is feeling anxious, advice for creating a relaxing environment is provided. In addition, when the subject is feeling tired, the relaxation proposal unit may propose taking a break. Thus, by proposing relaxation methods according to the subject's emotion, the system can support mental health.

The monitoring system may comprise an exercise recording unit configured to record the subject's exercise status. The exercise recording unit records the types and duration of exercises performed by the subject and can analyze the effects of exercise. For example, the content of walking or stretching performed by the subject is recorded to grasp the frequency and intensity of exercise. In addition, when a lack of exercise is detected, the exercise recording unit can provide advice to encourage exercise. Thus, the system can support the subject's exercise habits and contribute to maintaining health.

The monitoring system may comprise a hobby proposal unit configured to estimate the subject's emotion and propose hobby activities based on the estimated emotion. The hobby proposal unit estimates the subject's emotion and, for example, proposes new hobbies or activities when the subject is feeling bored. Hobbies related to fields of interest to the subject can also be proposed. In addition, when the subject is feeling lonely, the hobby proposal unit may propose hobbies that can be enjoyed with others. Thus, by proposing hobby activities according to the subject's emotion, the system can improve the quality of life.

The monitoring system may comprise an outing recording unit configured to record the subject's outing status. The outing recording unit records the places and times the subject goes out and can analyze the frequency and patterns of outings. For example, the places visited and the duration of stay are recorded to grasp the trends of outings. In addition, when outings are infrequent, the outing recording unit can provide advice to encourage going out. Thus, the system can grasp the subject's outing status and support a healthy lifestyle.

The monitoring system may comprise a mental health care unit configured to estimate the subject's emotion and provide mental health care based on the estimated emotion. The mental health care unit estimates the subject's emotion and, for example, provides counseling or information related to mental health when the subject is feeling depressed. When the subject is feeling anxious, advice to alleviate anxiety is provided. In addition, when the subject is feeling stressed, the mental health care unit may propose stress management methods. Thus, by providing mental health care according to the subject's emotion, the system can support mental health.

The monitoring system may comprise a hobby recording unit configured to record the subject's hobby activities. The hobby recording unit records the content and duration of hobby activities performed by the subject and can analyze hobby trends. For example, the content of handicrafts or gardening performed by the subject is recorded to grasp the frequency and types of hobbies. In addition, when hobby activities are infrequent, the hobby recording unit can propose new hobbies. Thus, the system can support the subject's hobby activities and improve the quality of life.

The following is a brief description of the processing flow of Example of the Embodiment.
Step 1: The speaking unit speaks to the subject. For example, the speaking unit speaks to the subject by voice at a fixed time every day. For example, “Good morning. What plans do you have today?” In this way, the subject can communicate with the generative AI on a daily basis.
Step 2: The conversation receiving unit receives a conversation from the subject spoken to by the speaking unit. For example, the conversation receiving unit receives the content of the subject's response to the speaking unit. For example, “I am planning to have lunch with a friend today.”
Step 3: The summarizing unit summarizes the conversation received by the conversation receiving unit. For example, the summarizing unit analyzes the content of the subject's conversation in real time using generative AI and extracts important information. The generative AI, for example, generates a summary such as “The subject is planning to have lunch with a friend.”
Step 4: The storage unit stores the content summarized by the summarizing unit. For example, the storage unit stores the summary generated by the generative AI on a server.
Step 5: The confirmation unit confirms the content stored by the storage unit. For example, the confirmation unit allows the service user to check the summary stored on the server at any desired timing.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned speaking unit, conversation receiving unit, summarizing unit, storage unit, confirmation unit, emotion analysis unit, anomaly detection unit, behavior monitoring unit, and keyword extraction unit is implemented by at least one of, for example, the smart device 14 and the data processing apparatus 12. For example, the speaking unit is implemented by a control unit 46A of the smart device 14 and speaks to the subject by voice at a fixed time every day. The conversation receiving unit is implemented by the control unit 46A of the smart device 14 and receives the subject's conversation. The summarizing unit is implemented by a specific processing unit 290 of the data processing apparatus 12 and summarizes the content of the conversation. The storage unit is implemented by the specific processing unit 290 of the data processing apparatus 12 and stores the summarized content on a server. The confirmation unit is implemented by the control unit 46A of the smart device 14 and confirms the stored content. The emotion analysis unit is implemented by the specific processing unit 290 of the data processing apparatus 12 and analyzes the subject's emotion. The anomaly detection unit is implemented by the specific processing unit 290 of the data processing apparatus 12 and detects anomalies by comparing with past conversation history. The behavior monitoring unit monitors the subject's behavior using a camera 42 or sensors of the smart device 14. The keyword extraction unit is implemented by the specific processing unit 290 of the data processing apparatus 12 and extracts important keywords from the content of the conversation. The correspondence between each unit and the device or control unit is not limited to the examples described above and various modifications are possible.

### Second Embodiment

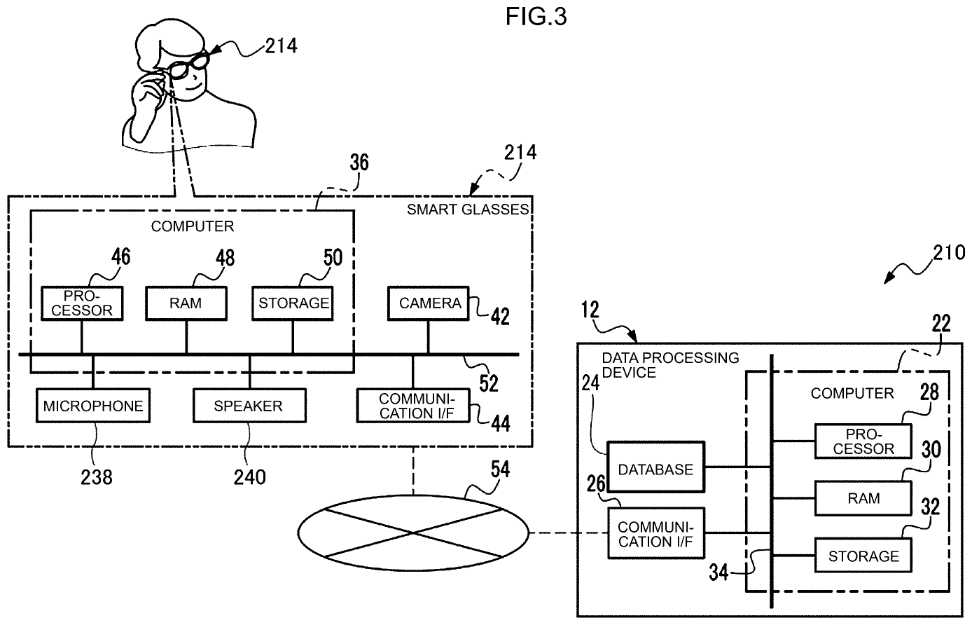

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

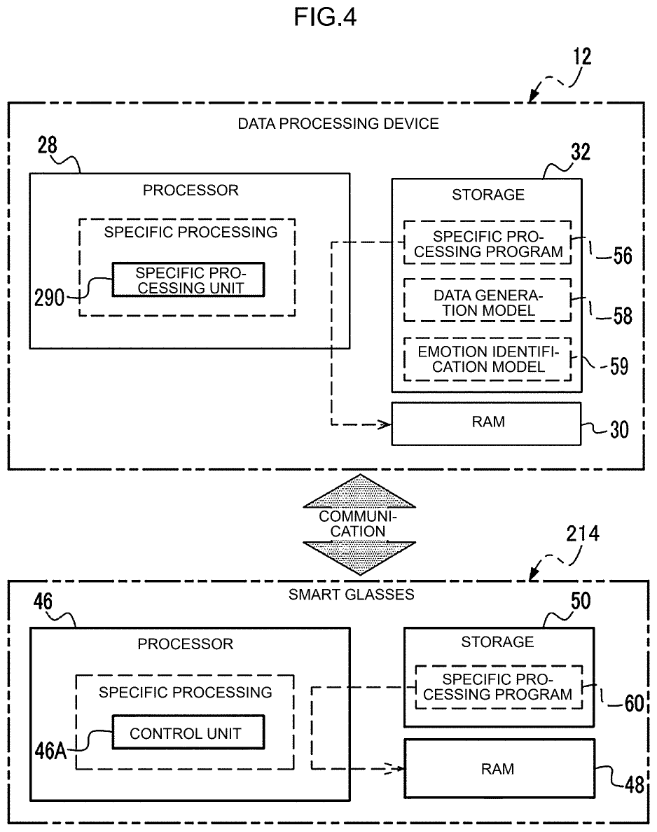

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned speaking unit, conversation receiving unit, summarizing unit, storage unit, confirmation unit, emotion analysis unit, anomaly detection unit, behavior monitoring unit, and keyword extraction unit is implemented by at least one of, for example, the smart glasses 214 and the data processing apparatus 12. For example, the speaking unit is implemented by a control unit 46A of the smart glasses 214 and speaks to the subject by voice at a fixed time every day. The conversation receiving unit is implemented by the control unit 46A of the smart glasses 214 and receives the subject's conversation. The summarizing unit is implemented by a specific processing unit 290 of the data processing apparatus 12 and summarizes the content of the conversation. The storage unit is implemented by the specific processing unit 290 of the data processing apparatus 12 and stores the summarized content on a server. The confirmation unit is implemented by the control unit 46A of the smart glasses 214 and confirms the stored content. The emotion analysis unit is implemented by the specific processing unit 290 of the data processing apparatus 12 and analyzes the subject's emotion. The anomaly detection unit is implemented by the specific processing unit 290 of the data processing apparatus 12 and detects anomalies by comparing with past conversation history. The behavior monitoring unit monitors the subject's behavior using a camera 42 or sensors of the smart glasses 214. The keyword extraction unit is implemented by the specific processing unit 290 of the data processing apparatus 12 and extracts important keywords from the content of the conversation. The correspondence between each unit and the device or control unit is not limited to the examples described above and various modifications are possible.

### Third Embodiment

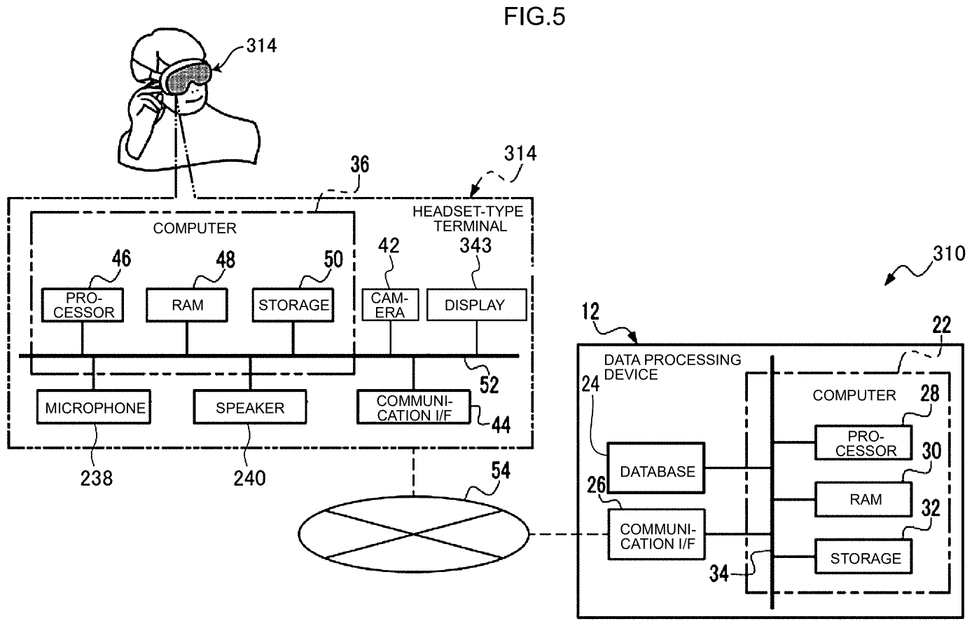

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

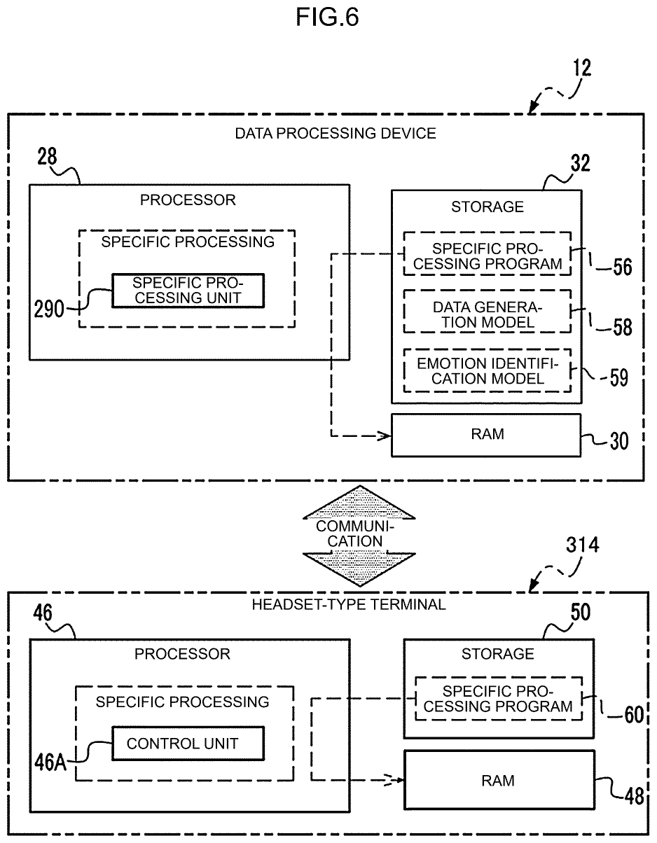

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned speaking unit, conversation receiving unit, summarizing unit, storage unit, confirmation unit, emotion analysis unit, anomaly detection unit, behavior monitoring unit, and keyword extraction unit is implemented by at least one of, for example, the headset-type terminal 314 and the data processing apparatus 12. For example, the speaking unit is implemented by a control unit 46A of the headset-type terminal 314 and speaks to the subject by voice at a fixed time every day. The conversation receiving unit is implemented by the control unit 46A of the headset-type terminal 314 and receives the subject's conversation. The summarizing unit is implemented by a specific processing unit 290 of the data processing apparatus 12 and summarizes the content of the conversation. The storage unit is implemented by the specific processing unit 290 of the data processing apparatus 12 and stores the summarized content on a server. The confirmation unit is implemented by the control unit 46A of the headset-type terminal 314 and confirms the stored content. The emotion analysis unit is implemented by the specific processing unit 290 of the data processing apparatus 12 and analyzes the subject's emotion. The anomaly detection unit is implemented by the specific processing unit 290 of the data processing apparatus 12 and detects anomalies by comparing with past conversation history. The behavior monitoring unit monitors the subject's behavior using a camera 42 or sensors of the headset-type terminal 314. The keyword extraction unit is implemented by the specific processing unit 290 of the data processing apparatus 12 and extracts important keywords from the content of the conversation. The correspondence between each unit and the device or control unit is not limited to the examples described above and various modifications are possible.

### Fourth Embodiment

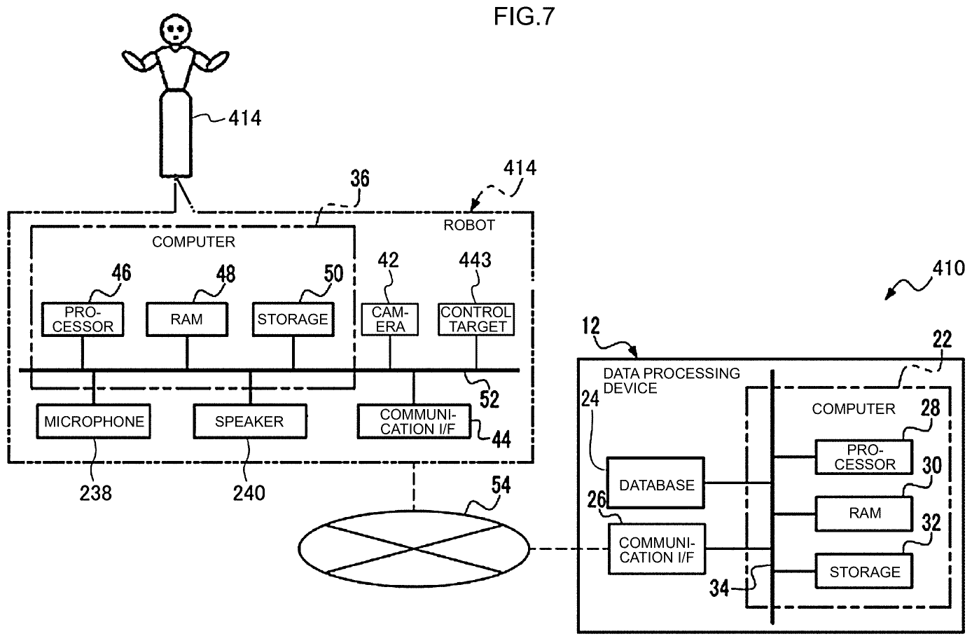

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

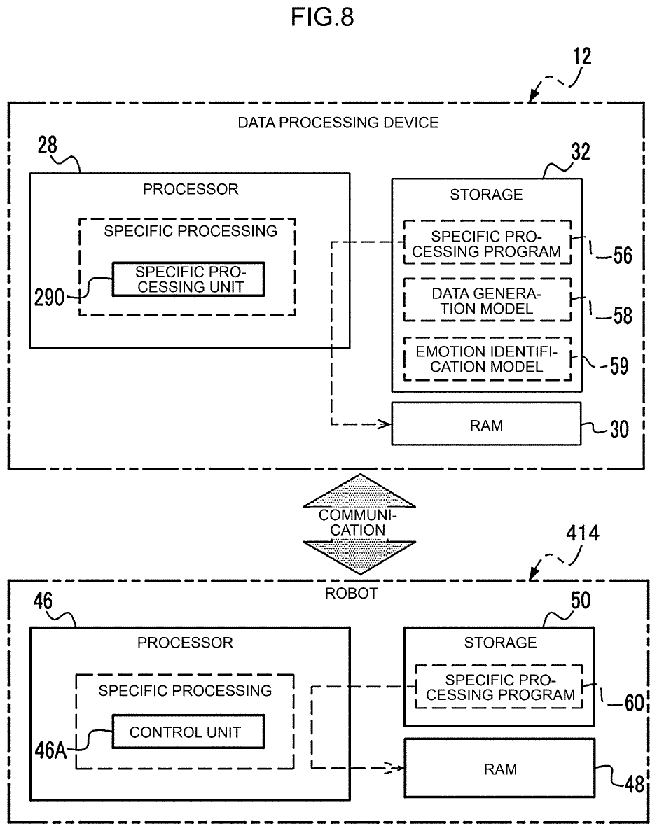

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned speaking unit, conversation receiving unit, summarizing unit, storage unit, confirmation unit, emotion analysis unit, anomaly detection unit, behavior monitoring unit, and keyword extraction unit is implemented by at least one of, for example, the robot 414 and the data processing apparatus 12. For example, the speaking unit is implemented by a control unit 46A of the robot 414 and speaks to the subject by voice at a fixed time every day. The conversation receiving unit is implemented by the control unit 46A of the robot 414 and receives the subject's conversation. The summarizing unit is implemented by a specific processing unit 290 of the data processing apparatus 12 and summarizes the content of the conversation. The storage unit is implemented by the specific processing unit 290 of the data processing apparatus 12 and stores the summarized content on a server. The confirmation unit is implemented by the control unit 46A of the robot 414 and confirms the stored content. The emotion analysis unit is implemented by the specific processing unit 290 of the data processing apparatus 12 and analyzes the subject's emotion. The anomaly detection unit is implemented by the specific processing unit 290 of the data processing apparatus 12 and detects anomalies by comparing with past conversation history. The behavior monitoring unit monitors the subject's behavior using a camera 42 or sensors of the robot 414. The keyword extraction unit is implemented by the specific processing unit 290 of the data processing apparatus 12 and extracts important keywords from the content of the conversation. The correspondence between each unit and the device or control unit is not limited to the examples described above and various modifications are possible.

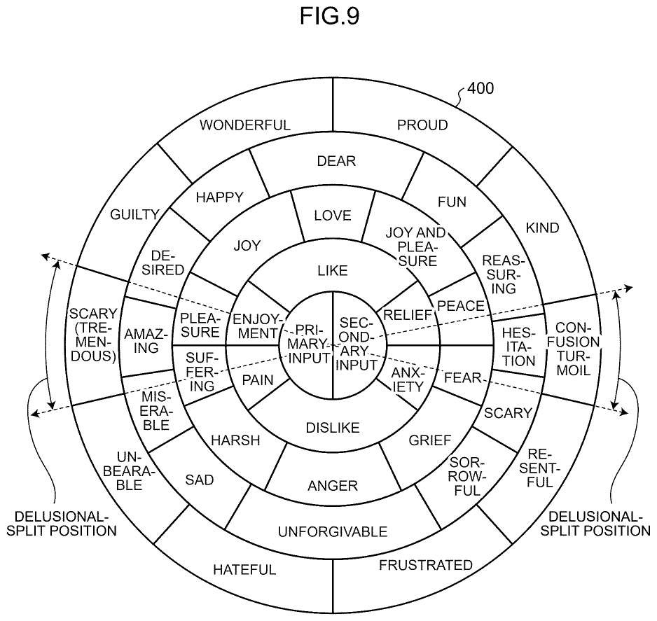

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

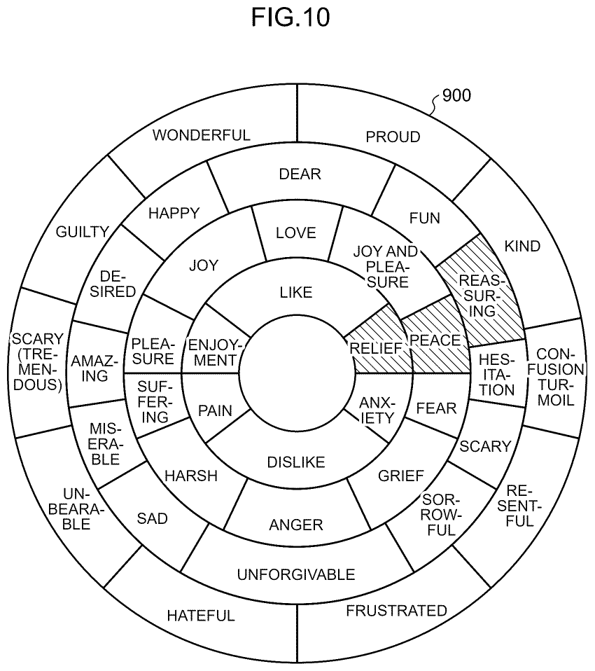

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: a speaking unit configured to speak to a subject; a conversation receiving unit configured to receive a conversation from the subject spoken to by the speaking unit; a summarizing unit configured to summarize the conversation received by the conversation receiving unit; a storage unit configured to store the content summarized by the summarizing unit; and a confirmation unit configured to confirm the content stored by the storage unit.
(Supplementary Note 2) The system according to Supplementary Note 1, further comprising an emotion analysis unit configured to perform emotion analysis.
(Supplementary Note 3) The system according to Supplementary Note 1, further comprising an anomaly detection unit configured to detect anomalies by comparing with past conversation history.
(Supplementary Note 4) The system according to Supplementary Note 1, further comprising a behavior monitoring unit configured to monitor the behavior of the subject.
(Supplementary Note 5) The system according to Supplementary Note 1, further comprising a keyword extraction unit configured to extract important keywords.
(Supplementary Note 6) The system according to Supplementary Note 1, wherein the speaking unit is configured to set a speaking time in accordance with the subject's daily rhythm.
(Supplementary Note 7) The system according to Supplementary Note 4, wherein the behavior monitoring unit is configured to issue an alert when the subject does not move for a certain period of time.
(Supplementary Note 8) The system according to Supplementary Note 2, wherein the emotion analysis unit is configured to analyze the subject's emotion from the content of the conversation.
(Supplementary Note 9) The system according to Supplementary Note 3, wherein the anomaly detection unit is configured to detect anomalies by comparing with past conversation history.
(Supplementary Note 10) The system according to Supplementary Note 1, wherein the speaking unit is configured to estimate the subject's emotion and adjust the content of the speech based on the estimated emotion.
(Supplementary Note 11) The system according to Supplementary Note 1, wherein the speaking unit is configured to analyze the subject's past conversation history and select the content of the speech.
(Supplementary Note 12) The system according to Supplementary Note 1, wherein the speaking unit is configured to select a topic based on the subject's current activity status when speaking.
(Supplementary Note 13) The system according to Supplementary Note 1, wherein the speaking unit is configured to estimate the subject's emotion and adjust the timing of the speech based on the estimated emotion.
(Supplementary Note 14) The system according to Supplementary Note 1, wherein the speaking unit is configured to select a highly relevant topic by considering the subject's geographic location when speaking.
(Supplementary Note 15) The system according to Supplementary Note 1, wherein the speaking unit is configured to analyze the subject's social media activity and select a relevant topic when speaking.
(Supplementary Note 16) The system according to Supplementary Note 1, wherein the conversation receiving unit is configured to estimate the subject's emotion and adjust the method of receiving the conversation based on the estimated emotion.
(Supplementary Note 17) The system according to Supplementary Note 1, wherein the conversation receiving unit is configured to refer to the subject's past conversation history and select an optimal receiving method when receiving a conversation.
(Supplementary Note 18) The system according to Supplementary Note 1, wherein the conversation receiving unit is configured to adjust the method of receiving the conversation based on the subject's current activity status when receiving a conversation.
(Supplementary Note 19) The system according to Supplementary Note 1, wherein the conversation receiving unit is configured to estimate the subject's emotion and determine the priority for receiving the conversation based on the estimated emotion.
(Supplementary Note 20) The system according to Supplementary Note 1, wherein the conversation receiving unit is configured to preferentially receive highly relevant conversations by considering the subject's geographic location when receiving a conversation.
(Supplementary Note 21) The system according to Supplementary Note 1, wherein the conversation receiving unit is configured to analyze the subject's social media activity and receive relevant conversations when receiving a conversation.
(Supplementary Note 22) The system according to Supplementary Note 1, wherein the summarizing unit is configured to estimate the subject's emotion and adjust the method of summarization based on the estimated emotion.
(Supplementary Note 23) The system according to Supplementary Note 1, wherein the summarizing unit is configured to adjust the level of detail of the summary based on the importance of the conversation when generating a summary.
(Supplementary Note 24) The system according to Supplementary Note 1, wherein the summarizing unit is configured to apply different summarization algorithms according to the category of the conversation when generating a summary.
(Supplementary Note 25) The system according to Supplementary Note 1, wherein the summarizing unit is configured to estimate the subject's emotion and adjust the length of the summary based on the estimated emotion.
(Supplementary Note 26) The system according to Supplementary Note 1, wherein the summarizing unit is configured to determine the priority of summarization based on the submission timing of the conversation when generating a summary.
(Supplementary Note 27) The system according to Supplementary Note 1, wherein the summarizing unit is configured to adjust the order of the summary based on the relevance of the conversation when generating a summary.
(Supplementary Note 28) The system according to Supplementary Note 1, wherein the storage unit is configured to estimate the subject's emotion and select the data to be stored based on the estimated emotion.
(Supplementary Note 29) The system according to Supplementary Note 1, wherein the storage unit is configured to refer to past stored data and select an optimal storage method when storing data.
(Supplementary Note 30) The system according to Supplementary Note 1, wherein the storage unit is configured to determine the priority of storage based on the importance of the data when storing data.
(Supplementary Note 31) The system according to Supplementary Note 1, wherein the storage unit is configured to estimate the subject's emotion and adjust the format of the data to be stored based on the estimated emotion.
(Supplementary Note 32) The system according to Supplementary Note 1, wherein the storage unit is configured to determine the priority of storage based on the submission timing of the data when storing data.
(Supplementary Note 33) The system according to Supplementary Note 1, wherein the storage unit is configured to adjust the order of storage based on the relevance of the data when storing data.
(Supplementary Note 34) The system according to Supplementary Note 1, wherein the confirmation unit is configured to estimate the subject's emotion and select the data to be confirmed based on the estimated emotion.
(Supplementary Note 35) The system according to Supplementary Note 1, wherein the confirmation unit is configured to refer to past confirmation data and select an optimal confirmation method when confirming data.
(Supplementary Note 36) The system according to Supplementary Note 1, wherein the confirmation unit is configured to determine the priority of confirmation based on the importance of the data when confirming data.
(Supplementary Note 37) The system according to Supplementary Note 1, wherein the confirmation unit is configured to estimate the subject's emotion and adjust the format of the data to be confirmed based on the estimated emotion.
(Supplementary Note 38) The system according to Supplementary Note 1, wherein the confirmation unit is configured to determine the priority of confirmation based on the submission timing of the data when confirming data.
(Supplementary Note 39) The system according to Supplementary Note 1, wherein the confirmation unit is configured to adjust the order of confirmation based on the relevance of the data when confirming data.
(Supplementary Note 40) The system according to Supplementary Note 2, wherein the emotion analysis unit is configured to estimate the subject's emotion from the content of the conversation and adjust the method of emotion analysis based on the estimated emotion.
(Supplementary Note 41) The system according to Supplementary Note 2, wherein the emotion analysis unit is configured to refer to past emotion data and select an optimal analysis method when performing emotion analysis.
(Supplementary Note 42) The system according to Supplementary Note 2, wherein the emotion analysis unit is configured to apply different analysis algorithms according to the category of the conversation when performing emotion analysis.
(Supplementary Note 43) The system according to Supplementary Note 2, wherein the emotion analysis unit is configured to estimate the subject's emotion from the content of the conversation and adjust the method of displaying the analysis result based on the estimated emotion.
(Supplementary Note 44) The system according to Supplementary Note 2, wherein the emotion analysis unit is configured to determine the priority of analysis based on the submission timing of the conversation when performing emotion analysis.
(Supplementary Note 45) The system according to Supplementary Note 2, wherein the emotion analysis unit is configured to adjust the order of analysis based on the relevance of the conversation when performing emotion analysis.
(Supplementary Note 46) The system according to Supplementary Note 3, wherein the anomaly detection unit is configured to detect anomalies by comparing with past conversation history and adjust the method of anomaly detection based on the detected anomalies.
(Supplementary Note 47) The system according to Supplementary Note 3, wherein the anomaly detection unit is configured to refer to past anomaly data and select an optimal detection method when performing anomaly detection.
(Supplementary Note 48) The system according to Supplementary Note 3, wherein the anomaly detection unit is configured to apply different detection algorithms according to the category of the conversation when performing anomaly detection.
(Supplementary Note 49) The system according to Supplementary Note 3, wherein the anomaly detection unit is configured to detect anomalies by comparing with past conversation history and adjust the method of displaying the detection result based on the detected anomalies.
(Supplementary Note 50) The system according to Supplementary Note 3, wherein the anomaly detection unit is configured to determine the priority of detection based on the submission timing of the conversation when performing anomaly detection.
(Supplementary Note 51) The system according to Supplementary Note 3, wherein the anomaly detection unit is configured to adjust the order of detection based on the relevance of the conversation when performing anomaly detection.
(Supplementary Note 52) The system according to Supplementary Note 4, wherein the behavior monitoring unit is configured to monitor the behavior of the subject and adjust the monitoring method based on the monitored behavior.
(Supplementary Note 53) The system according to Supplementary Note 4, wherein the behavior monitoring unit is configured to refer to past behavior data and select an optimal monitoring method when performing behavior monitoring.
(Supplementary Note 54) The system according to Supplementary Note 4, wherein the behavior monitoring unit is configured to apply different monitoring algorithms according to the category of the behavior when performing behavior monitoring.
(Supplementary Note 55) The system according to Supplementary Note 4, wherein the behavior monitoring unit is configured to monitor the behavior of the subject and adjust the method of displaying the monitoring result based on the monitored behavior.
(Supplementary Note 56) The system according to Supplementary Note 4, wherein the behavior monitoring unit is configured to determine the priority of monitoring based on the submission timing of the behavior when performing behavior monitoring.
(Supplementary Note 57) The system according to Supplementary Note 4, wherein the behavior monitoring unit is configured to adjust the order of monitoring based on the relevance of the behavior when performing behavior monitoring.
(Supplementary Note 58) The system according to Supplementary Note 5, wherein the keyword extraction unit is configured to extract important keywords from the content of the conversation and adjust the extraction method based on the extracted keywords.
(Supplementary Note 59) The system according to Supplementary Note 5, wherein the keyword extraction unit is configured to refer to past keyword data and select an optimal extraction method when performing keyword extraction.
(Supplementary Note 60) The system according to Supplementary Note 5, wherein the keyword extraction unit is configured to apply different extraction algorithms according to the category of the conversation when performing keyword extraction.
(Supplementary Note 61) The system according to Supplementary Note 5, wherein the keyword extraction unit is configured to extract important keywords from the content of the conversation and adjust the method of displaying the keywords based on the extracted keywords.
(Supplementary Note 62) The system according to Supplementary Note 5, wherein the keyword extraction unit is configured to determine the priority of extraction based on the submission timing of the conversation when performing keyword extraction.
(Supplementary Note 63) The system according to Supplementary Note 5, wherein the keyword extraction unit is configured to adjust the order of extraction based on the relevance of the conversation when performing keyword extraction.

What is claimed is:

1. A system comprising:
circuitry configured to:
generate, using a speech synthesis model, audio data encoding a query directed to a client terminal communicatively coupled to the system via a packet-switched network;
transmit the audio data to the client terminal;
receive, from the client terminal, response data encoding a spoken response to the query;
generate, using a text generation model, summary data by summarizing content extracted from the response data;
store the summary data in association with a timestamp; and
transmit, to a remote terminal, confirmation data indicating the stored summary data.

2. The system according to claim 1, wherein the circuitry is further configured to compute an emotion value by applying an emotion identification model to at least one of: acoustic features extracted from the response data, or text features extracted from the response data.

3. The system according to claim 1, wherein the circuitry is further configured to compute an anomaly score by comparing the summary data with historical summary data stored in association with prior timestamps.

4. The system according to claim 1, wherein the circuitry is further configured to receive sensor data from the client terminal and detect a behavioral state based on the sensor data.

5. The system according to claim 1, wherein the circuitry is further configured to extract keywords from the response data using the text generation model.

6. The system according to claim 1, wherein the circuitry is configured to generate the audio data at a time determined based on activity pattern data associated with the client terminal.

7. The system according to claim 4, wherein the circuitry is configured to generate an alert when the sensor data indicates an absence of motion for a threshold duration.

8. The system according to claim 2, wherein the circuitry is configured to compute the emotion value based on context vectors generated by tokenizing text extracted from the response data.

9. The system according to claim 3, wherein the circuitry is configured to compute the anomaly score using cosine similarity between a current summary vector and historical summary vectors.

10. The system according to claim 2, wherein the circuitry is configured to adjust content of the audio data based on the emotion value.

11. The system according to claim 1, wherein the circuitry is configured to select a topic for the query based on historical response data associated with the client terminal.

12. The system according to claim 4, wherein the circuitry is configured to select a topic for the query based on the behavioral state.

13. The system according to claim 2, wherein the circuitry is configured to adjust a transmission time for the audio data based on the emotion value.

14. The system according to claim 1, wherein the circuitry is configured to select a topic for the query based on geographic location data received from the client terminal.

15. The system according to claim 3, wherein the circuitry is configured to transmit an alert to the remote terminal when the anomaly score exceeds a threshold.

16. The system according to claim 1, wherein the circuitry is configured to adjust a level of detail of the summary data based on an importance score computed for the response data.

17. The system according to claim 1, wherein the circuitry is configured to apply different summarization parameters based on a category assigned to the response data.

18. A system comprising:
a communication interface configured to communicate with a client terminal via a packet-switched network;
a processor;
a random-access memory;
a memory storing a speech synthesis model, a text generation model, and an emotion identification model; and
circuitry configured to:
generate, using the speech synthesis model, audio data encoding a query;
transmit, via the communication interface, the audio data to the client terminal;
receive, via the communication interface, response data encoding a spoken response from the client terminal;
compute an emotion value by applying the emotion identification model to features extracted from the response data;
generate, using the text generation model, summary data by summarizing content extracted from the response data;
store the summary data in association with a timestamp and the emotion value; and
transmit, via the communication interface, confirmation data indicating the stored summary data to a remote terminal.

19. The system according to claim 18, wherein the circuitry is further configured to compute an anomaly score by comparing the summary data with historical summary data, and transmit an alert to the remote terminal when the anomaly score exceeds a threshold.

20. A method performed by circuitry of a system, the method comprising:
generating, using a speech synthesis model, audio data encoding a query directed to a client terminal communicatively coupled to the system via a packet-switched network;
transmitting the audio data to the client terminal;
receiving, from the client terminal, response data encoding a spoken response to the query;
generating, using a text generation model, summary data by summarizing content extracted from the response data;
storing the summary data in association with a timestamp; and
transmitting, to a remote terminal, confirmation data indicating the stored summary data.
